(19) United States
(12) Patent Application Publication
(10) Pub. No.: US 2010/0112600 A1
(43) Pub. Date: May 6, 2010

(54) Methods and compositions for modulating synapse formation

(76) Inventors: Azard Bonni, Brookline, MA (US); Aryaman Shalizi, Menlo Park, CA (US); Brice Gaudilliere, Cambridge, MA (US)

Correspondence Address: KEOWN & ZUCCHERO, LLP, 500 WEST CUMMINGS PARK, SUITE 1200, WOBURN, MA, 01801, US

(21) Appl. No.: 12/309,291

(22) PCT Filed: Jul. 13, 2007
(86) PCT No.: PCT/US2007/015959

§ 371 Date: Jan. 6, 2010

Related U.S. Application Data
(60) Provisional application No. 60/831,017, filed on Jul. 13, 2006.

(51) Int. Cl. G01N 33/53; C12N 5/079

(52) U.S. Cl. 435/7.21; 435/375

(57) ABSTRACT

The invention provides methods of modulating synapse formation. Methods for identifying agents to modulate synapse formation are also disclosed.

### STATEMENT AS TO FEDERALLY SPONSORED RESEARCH

This invention was funded in part by the U.S. Government under grant number NS41021 awarded by the National Institutes of Health. The Government has certain rights in the invention.

### BACKGROUND OF THE INVENTION

Neuronal synapses are the sites that allow information to be transmitted between one neuron to the other. Synapses therefore play a key role during neural development and regeneration as well as neural plasticity. Growing evidence suggests that synaptic abnormalities are involved in the pathogenesis of various neurological disorders such as strokes, Alzheimer's disease, mental retardation, and other psychiatric disorders. Accordingly, a better understanding of the process of synapse formation would help in designing better therapeutic modalities.

### SUMMARY OF THE INVENTION

The present invention provides methods for modulating, i.e. increasing or decreasing, neuronal synapse formation by modulating the activity of the transcriptional factor myocyte enhancer factor 2 (MEF2) (e.g., MEF2A), MEF2C, MEF2D, dMEF2, CeMEF2, Activating transcription factor 6 beta (ATF6), Estrogen related receptor alpha (ERR1), Estrogen related receptor beta (ERR2), Estrogen related receptor gamma (ERR3), Erythroblastosis virus E26 oncogene homolog 1 (ETS1), Forkhead box protein C2 (FOXC2), Gata binding factor 1 (GATA-1), Heat shock factor 1 (HSF1), HSF4, MLL3, Myeloblastosis oncogene homolog (MYB), Nuclear receptor coactivator 2 (NCOA2), Nuclear receptor corepressor 1 (NCOR1), Peroxisome proliferative activated receptor gamma (PPARg), SMAD nuclear interacting protein 1 (SNIP1), SRY-box containing protein 3 (SOX3), SOX8, SOX9, Sterol regulatory element-binding transcription factor 2 (SREBP2), or Thyroid hormone receptor beta-1 (THRB1). The activity of MEF2 is modulated by post-translational modifications (e.g., amino acid phosphorylation, acetylation, or sumoylation) within the sumoylation acetylation switch (SAS) peptide motifs. Methods for screening agents useful for modulating synapse formation are also described.

A method of identifying a candidate compound useful for modulating synapse formation (e.g., dendrite formation or maturation such as dendritic claw differentiation) involves the steps of: (a) contacting a cell expressing a sumoylation-acetylation switch (SAS) peptide motif gene with a candidate compound; and (b) measuring the level of Serine 408 (Ser408) phosphorylation of the SAS peptide motif in the cell. A modulation, i.e. an increase or a decrease in the phosphorylation level of Ser408 in the presence of the compound compared to such levels in the absence of the compound indicates that the compound modulates synapse formation. If desired, step (b) includes measuring the level of sumoylation, acetylation, or both at the Lys403 residue in the SAS motif peptide. Detection of sumoylation or an increase of sumoylation indicates that the candidate compound is useful to promote or increase synapse formation, number or differentiation, whereas a decrease indicates that the compound reduces synapse formation, number, or extent of differentiation.

Another method of identifying a compound useful for modulating synapse formation involves: (a) contacting a cell expressing a SAS peptide motif gene with a candidate compound; and (b) measuring the level of sumoylation at lysine 403 (Lys403) in the SAS peptide motif, such that an increase or decrease in the sumoylation levels at Lys403 relative to a control identifies the candidate compound as being useful for modulating synapse formation, number, or extent of differentiation. Optionally, step (b) includes measuring the level of phosphorylation at Ser408 or the level of acetylation at Lys403 residue in the SAS motif peptide.

Yet another method of identifying a compound useful for modulating synapse formation involves: (a) contacting a cell expressing a SAS peptide motif gene with a candidate compound; and (b) measuring the level of acetylation at lysine 403 (Lys403) in the SAS peptide motif, such that an increase or decrease in the acetylation levels at Lys403 relative to a control identifies the candidate compound as being useful for modulating synapse formation, number, or extent of differentiation. Optionally, step (b) includes measuring the level of phosphorylation at Ser408 or the level of sumoylation at Lys403 residue in the SAS motif peptide.

In all foregoing aspects of the invention, a compound that promotes dendritic claw formation, thereby increasing synapse formation or maturation is a compound that increases phosphorylation of Ser408, increases sumoylation at Lys403, or reduces acetylation at Lys403. Conversely, a compound that reduces synaptic function is a compound that reduces phosphorylation of Ser408, reduces sumoylation at Lys403, or increases acetylation at Lys403. Desirably, the SAS peptide motif gene is a MEF2A gene. Optionally, the SAS peptide motif gene is a SAS peptide motif fusion gene. The methods described above employ any mammalian cell including a human or a rodent cell. Cell types amenable to the screening methods described herein also include neural cells, such as cerebellar granule neurons. Optionally, step (b) in any of the above methods includes measuring the expression or activity level of Nur77. For example, the level of Nur77 mRNA is measured. Preferably, the cell is not a hippocampal neuron cell.

The invention also features a method of modulating synapse formation by contacting a neural cell (e.g., granule neuron) with an agent that modulates the level of Ser408 phosphorylation in the SAS peptide motif of MEF2A. Exemplary agents that increase the level of Ser408 phosphorylation in the SAS peptide motif of MEF2A, thereby increasing synapse number or formation, are phosphatase inhibitors, such as cyclosporin A and FK506. Useful agents include those that increase the activity of a kinase.

Another method to modulate synapse formation involves contacting a neural cell with an agent that modulates the level of acetylation at Lys403 in the SAS peptide motif of MEF2A. Agents that reduce the level of acetylation, thereby increasing synapse formation, include nimodipine, curcumin and its derivatives, HAT inhibitors, and VSCC or calcineurin inhibitors such as CsA. Agents that increase the level of acetylation thereby reducing synapse formation include agents that reduce the expression or activity level of a histone deacetylase (HDAC) (e.g., class I HDAC, class II HDAC, and class III HDAC), trichostatin A, suberoylanilide hydroxamic acid (SAHA), pyroxamide, apicidin, depudecin, depsipeptide, oxamflatin, CI-994 (N-acetyl dinaline), m-Carboxy cinnamic acid bishydroxamic acid (CBHA), scriptaid, trapoxin, TPX-HA analogue (CHAP), and sirtinol. Other exemplary agents are agents that increase the expression or activity level of a histone acetyltransferase.

Yet another method to modulate synapse formation involves contacting a neural cell with an agent that modulates the level of sumoylation at Lys403 in the SAS peptide motif of MEF2A. Optionally, the agent increases the level of sumoylation in the cell, thereby increasing synapse formation. An exemplary agent that increases the level of sumoylation in the cell is PIASx, or a compound that augments PIASx expression or activity. Optionally, standard gene therapy vectors are used for local administration of DNA to modulate the level of sumoylation in the cell. Exogenous DNA encoding agents that increase the level of sumoylation are optionally administered to increase the level of sumoylation in the cell, thereby increasing synapse formation. For example, Exogenous DNA encoding PIASx is administered. Alternatively, the agent decreases the level of sumoylation in the cell, thereby decreasing synapse formation. Optionally, the expression of agents that modulate the level of sumoylation in the cell is reduced or knocked-down using small interfering RNA (siRNA), microRNA (miRNA), antisense, hairpin RNA, or RNAi strategies. Alternatively, any mechanism that interferes with transcription or translation is used to knockdown the expression of an agent that modulates the level of sumoylation in the cell. An agent that reduces the level of sumoylation includes, for example, an agent that reduces the expression or activity level of a SUMO protease or isopeptidase, Ubc9, or SUMO E3 ligase. For example, the agent is N-ethylmaleimide. Optionally, the agent is a SUMO-removing isopeptidase or an isopeptidase inhibitor. Agents that increase the level of sumoylation in the cell, thereby increasing synapse formation, also reduce the level of acetylation at Lys403 and include agents that reduce the expression or activity level of a histone acetyl transferase enzyme, such as curcumin and its derivatives as well as HAT inhibitors. Agents that increase the level of sumoylation include agents that increase the expression of the SUMO-conjugating enzyme Ub9 or the expression of a SUMO E3 ligase. Other agents that increase the level of sumoylation at Lys403 include nimodipine as well as voltage-sensitive calcium channel (VSCC) or calcineurin inhibitors, including cyclosporin A (CsA).

Desirably, the agent that modulates synapse formation is a small molecule inhibitor or an RNA interfering agent. A small molecule inhibitor is a compound that is less than 2000 daltons in mass. The molecular mass of the inhibitory compounds is preferably less than 1000 daltons, more preferably less than 600 daltons, e.g., the compound is less than 500 daltons, 400 daltons, 300 daltons, 200 daltons, or 100 daltons. Preferably, the inhibitor is not a peptide or proteinaceous in nature.

Another method of modulating synapse formation involves contacting a neural cell with an agent that modulates the activity of a SUMO ligase in the cell. For example, the SUMO ligase is a SUMO E3 ligase, such as PIASx. Optionally, the agent increases the expression or activity of a SUMO E3 ligase, such as PIASx, in the cell, thereby increasing synapse formation. Alternatively, the agent decreases the expression or activity of a SUMO E3 ligase in the cell, thereby decreasing synapse number or formation.

A method for identifying a candidate compound that modulates association of PIASx with MEF2A involves: (a) contacting a cell expressing a MEF2A gene with a candidate compound; and (b) measuring the level of sumoylation at lysine 403 (Lys403) in the MEF2A gene in the cell, such that an increase or decrease in the sumoylation levels in the presence of the compound compared to that in the absence of the compound indicates that the compound modulates association of PIASx with MEF2A. Optionally, the agent increases the association of PIASx with MEF2A in the cell, thereby increasing synapse formation. Alternatively, the agent decreases the association of PIASx with MEF2A in the cell, thereby decreasing synapse formation, number or extent of differentiation.

Another method for identifying a candidate compound that modulates association of PIASx with MEF2A involves: (a) contacting a cell expressing a MEF2A gene with a candidate compound and (b) measuring the association of PIASx with MEF2A in the cell, such that an increase or decrease in the association levels in the presence of the compound compared to that in the absence of the compound indicates that the compound modulates association of PIASx with MEF2A. Optionally, the agent increases the association of PIASx with MEF2A in the cell, thereby increasing synapse formation, number or extent of differentiation. Alternatively, the agent decreases the association of PIASx with MEF2A in the cell, thereby decreasing synapse formation.

A method for identifying a candidate compound that modulates the enzymatic activity of PIASx involves: (a) contacting a cell expressing PIASx with a candidate compound and (b) measuring the enzymatic activity of PIASx in the cell, such that an increase or decrease in enzymatic activity levels in the presence of the compound compared to that in the absence of the compound indicates that the compound modulates enzymatic activity of PIASx.

The methods described herein are used to reduce a symptom of a disorder selected from the group consisting of Alzheimer's disease, Parkinson's disease, stroke, multiple sclerosis, spinal cord injuries, depression, schizophrenia, anxiety, Huntington's Disease, ALS, mental retardation (Down syndrome or Fragile X syndrome) and spinal muscular atrophy.

The invention includes the use of an inhibitor of MEF2-dependent transcription in the manufacture of a medicament for increasing synapse formation, number, or extent of differentiation.

The invention also includes the use of a PIASx activator in the manufacture of a medicament for increasing synapse formation, number, or extent of differentiation.

By “modulating” the level of association, phosphorylation, acetylation, or sumoylation of an amino acid in a polypeptide is meant to increase or reduce the level of association, phosphorylation, acetylation, or sumoylation of an amino acid in a polypeptide compared to such level in an untreated control. These levels are modulated by at least 10%, 20%, 30%, 40%, 50%, 60%, 70%, 80%, 90%, or 100%, relative to an untreated control. Synapse formation is preferably modulated, i.e., increased or reduced by at least 10%, 20%, 30%, 40%, 50%, 60%, 70%, 80%, 90%, or 100%, relative to an untreated control.

The methods are useful to promote dendrite development as well as synapse formation and maturation to treat or reduce the severity of CNS injuries as well as psychiatric and neurologic disorders, such as Alzheimer'disease, Parkinson's disease, stroke, multiple sclerosis, spinal cord injuries, depression, schizophrenia, anxiety, Huntington's Disease, ALS, mental retardation (Down syndrome or Fragile X syndrome) or spinal muscular atrophy. As compared with an equivalent untreated control, symptoms are reduced by (or the degree of prevention is reduced by) at least 5%, 10%, 20%, 40%, 50%, 60%, 70%, 80%, 90%, 95%, or 100% as measured by any standard technique. Diagnosis of these disorders is well known in the art and involves, for example, the detection of symptoms associated with the disorder (e.g., tremors, impaired cognition, seizures, memory loss, headaches, and agitation), CAT scans, and Magnetic resonance imaging. One in the art will understand that these patients may have been subjected to the same standard tests as described above or may have been identified, without examination, as one at high risk due to the presence of one or more risk factors (e.g., family history or genetic predisposition).

As used herein, “sumoylation-acetylation switch” or “SAS” peptide motif refers to the an amino acid sequence that acts as a phosphorylation-regulated sumoylation-acetylation switch within a MEF2 polypeptide. The modifications of MEF2A required for postsynaptic differentiation occur within this SAS peptide motif that is conserved in all major MEF2 isoforms, as well as several other transcription factor families. The SAS peptide motif is substantially identical to the naturally occurring SAS peptide motif in the MEF2A gene (Accession numbers NP_{—}005578 (human) [amino acids 402-409] AAH53871 [amino acids 313-320] (human), AAH13437 (human) [amino acids 394-401], AAH96598 (mouse) [amino acids 394-401], NP_{—}001028885 (mouse) [amino acids 400-407], and NP_{—}001014057 (rat) [amino acids 394-401]), the sequences of which are hereby incorporated by reference). According to this invention, synapse formation is modulated when the level of phosphorylation at Ser408 or the level of sumoylation or acetylation at Lys403 within the SAS peptide motif of an MEF2A gene is modulated by at least 5%, 10%, 20%, 30%, 40%, 50%, 60%, 70%, 80%, 90%, or 100% compared to control levels as measured by any standard method. A SAS peptide gene is a nucleic acid that encodes a SAS peptide, such as those listed above. A SAS fusion gene includes a MEF2 promoter and/or all or part of an SAS peptide coding region operably linked to a second, heterologous nucleic acid sequence. In preferred embodiments, the second, heterologous nucleic acid sequence is a reporter gene, that is, a gene whose expression may be assayed; reporter genes include, without limitation, those encoding glucuronidase (GUS), luciferase, chloramphenicol transacetylase (CAT), green fluorescent protein (GFP), alkaline phosphatase, and beta-galactosidase.

By “purified antibody” is meant antibody which is at least 60%, by weight, free from proteins and naturally occurring organic molecules with which it is naturally associated. Preferably, the preparation is at least 75%, more preferably 90%, and most preferably at least 99%, by weight, antibody, e.g., a SAS specific antibody. A purified antibody may be obtained, for example, by affinity chromatography using recombinantly-produced protein or conserved motif peptides and standard techniques. A specific antibody recognizes and binds an antigen or antigenic domain such as a SAS peptide but that does not substantially recognize and bind other molecules in a sample, e.g., a biological sample, that naturally includes protein or domains of a target protein. Neutralizing antibodies interfere with any of the biological activity of an SAS peptide within the MEF2 polypeptide (e.g., the ability to modulate synapse formation). The neutralizing antibody may reduce SAS peptide signaling activity by, preferably 50%, more preferably by 70%, and most preferably by 90% or more.

By “substantially identical,” when referring to a protein or polypeptide, is meant a protein or polypeptide exhibiting at least 75%, but preferably 85%, more preferably 90%, most preferably 95%, or even 99% identity to a reference amino acid sequence. For proteins or polypeptides, the length of comparison sequences will generally be at least 20 amino acids, preferably at least 30 amino acids, more preferably at least 40 amino acids, and most preferably 50 amino acids or the full length protein or polypeptide. Nucleic acids that encode such “substantially identical” proteins or polypeptides constitute an example of “substantially identical” nucleic acids; it is recognized that the nucleic acids include any sequence, due to the degeneracy of the genetic code, that encodes those proteins or polypeptides. In addition, a “substantially identical” nucleic acid sequence also includes a polynucleotide that hybridizes to a reference nucleic acid molecule under high stringency conditions.

By “high stringency conditions” is meant any set of conditions that are characterized by high temperature and low ionic strength and allow hybridization comparable with those resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1× Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well known by those skilled in the art of molecular biology. See, e.g., F. Ausubel et al., Current Protocols in Molecular Biology, John Wiley & Sons, New York, N.Y., 1998, hereby incorporated by reference.

By “substantially pure” is meant a nucleic acid, polypeptide, or other molecule that has been separated from the components that naturally accompany it. Typically, the polypeptide is substantially pure when it is at least 60%, 70%, 80%, 90%, 95%, or even 99%, by weight, free from the proteins and naturally-occurring organic molecules with which it is naturally associated. For example, a substantially pure polypeptide may be obtained by extraction from a natural source, by expression of a recombinant nucleic acid in a cell that does not normally express that protein, or by chemical synthesis. The term “isolated DNA” is meant DNA that is free of the genes which, in the naturally occurring genome of the organism from which the given DNA is derived, flank the DNA. Thus, the term “isolated DNA” encompasses, for example, cDNA, cloned genomic DNA, and synthetic DNA.

By “an effective amount of a synapse-modulating compound” is meant an amount of a compound, alone or in a combination, required to promote or reduce synapse formation or maturation in a mammal. The effective amount of active compound(s) varies depending upon the route of administration, age, body weight, and general health of the subject. Ultimately, the attending physician or veterinarian will decide the appropriate amount and dosage regimen.

A candidate compound is a chemical, be it naturally-occurring or artificially-derived that is tested using screening methods described herein to identify synapse modulating activity. Candidate compounds may include, for example, peptides, polypeptides, synthetic organic molecules, naturally occurring organic molecules, nucleic acid molecules, peptide nucleic acid molecules, and components and derivatives thereof. The term “pharmaceutical composition” is meant any composition, which contains at least one therapeutically or biologically active agent and is suitable for administration to the patient. Any of these formulations can be prepared by well-known and accepted methods of the art. See, for example, Remington: The Science and Practice of Pharmacy, 20^{th} edition, (ed. A. R. Gennaro), Mack Publishing Co., Easton, Pa., 2000.

The present invention provides significant advantages over standard therapies for treatment, prevention, and reduction, or alternatively, the alleviation of one or more symptoms associated with aberrant or faulty synapse formation or neuronal damage. In addition, because the methods specifically target dendrites avoiding side-effects associated with broad-based drug approaches, the screening methods identify therapeutic compounds that modify the injury process, rather than merely mitigating the symptoms.

Cited publications including sequences defined by GENBANK™ accession numbers are incorporated herein by reference. Other features, objects, and advantages of the invention will be apparent from the description of the drawings.

### BRIEF DESCRIPTION OF THE FIGURES

FIG. 1A is a diagram of developing rat cerebellar cortex: external granule (EGL), molecular (ML), Purkinje cell (PL), and internal granule (IGL) layers. Granule neurons (GN) elaborate dendritic claws that contact mossy fiber (MF) axons.

FIGS. 1B-1D are representative images of GFP-positive granule neurons within transfected cerebellar slices. Asterisk and arrowheads indicate the cell body and axons, respectively, in (B). Numbers in (C) indicate dendritic claws shown at higher magnification in the right panels; arrows indicate dendritic claws. Neurons immunostained for GFP and PSD95 in (D). The claw and the shaft of the dendrite are indicated, and arrowheads indicate PSD95-positive puncta within the claw (see bar graph). PSD95 puncta density is significantly higher in the claws than in the shaft of dendrites (p<0.001, t-test; n=22).

FIG. 1E is a series of images of immunofluorescent cell stains, a series of immunoblots, and a bar graph. In the upper right panel, lysates of 293T cells transfected with control U6 or U6/mef2a plasmid together with expression plasmids for MEF2A encoded by wild type cDNA (MEF2A-WT) or RNAi resistant cDNA (MEF2A-Res) were immunoblotted for MEF2A. In the left panels, cerebellar slices were transfected with the control U6 or U6/mef2a plasmid together with expression plasmids encoding MEF2A-WT or MEF2A-Res and GFP and analyzed for dendritic claws. In the lower right panel, quantification indicates that MEF2A knockdown significantly reduces dendritic claw number, an effect that is reversed by MEF2A-Res but not MEF2A-WT (p<0.001, ANOVA; n=262). Scale bar=5 μm (C) and 3 μm (D and E).

FIG. 2A is a series of immunofluorescent cell stains. The cerebellum of P3 rat pups was injected and electroporated with the U6-cmvGFP plasmid. Representative electroporated granule neurons with cell bodies in the IGL are shown. Scale bar=50 μm (left panel); 10 μm (middle panel); 5 μm (right panel). Asterisk, arrowheads and arrows respectively indicate the cell body, parallel fibers and dendritic claws of granule neurons.

FIGS. 2B and 2C are immunofluorescent cell stains and a bar graph. Sections of U6-cmvGFP-electroporated cerebellum were immunostained with antibodies to GFP and PSD95 (B) or synaptophysin (C). An image of a dendritic claw analyzed as in FIG. 1D is shown, and PSD95 density is quantified in the bar graph. PSD95 puncta density is significantly higher in the claw than in the shaft of dendrites (p<0.001, t-test; n=3).

FIG. 2D is a series of immunofluorescent stains and a bar graph. Representative granule neuron dendrites from cerebella transfected with the control U6-cmvGFP or U6/mef2a-cmvGFP plasmid. The bar graph shows that MEF2A knockdown significantly reduces dendritic claw number in vivo (p<0.01, t-test; n=124). Scale bar=5 μm (B-D).

FIG. 3A is a series of immunoblots showing activity-dependent dephosphorylation of MEF2A Ser408 in neurons. Lysates of granule neurons were depolarized with 25 mM KCl were immunoblotted for MEF2A, phospho-Ser408 MEF2A or ERK1/2. Nim=Nimodipine, 20 μM. CsA=Cyclosporin A, 4 μM.

FIG. 3B is a series of immunoblots showing that calcineurin dephosphorylates MEF2A Ser408. Lysates of 293T cells transfected with MEF2A and HA-tagged constitutively active calcineurin subunit A (HA-CnA*) or control vector were immunoblotted for MEF2A, MEF2ApS408, or HA. NS=non-specific band.

FIG. 3C is a series of immunoblots showing that MEF2A Lys403 is both sumoylated and acetylated. Lysates and GAL4-immunoprecipitates of 293T cells transfected with wild type, K403R or E405D mutant G4-MEF2A and HA-SUMO1 were immunoblotted for HA, acetyl-lysine (AcK) or MEF2A. Both K403R and E405D mutations block MEF2A sumoylation, indicating that Lys403 is a bona-fide SUMO acceptor site.

FIG. 3D is a series of immunoblots showing that calcineurin inhibits sumoylation and promotes acetylation of MEF2A. Cells transfected with G4-MEF2A and HA-CnA* were analyzed as in FIG. 3C.

FIG. 3E is a series of immunoblots showing that Ser408 is required for MEF2A sumoylation. Cells transfected with wild type, K403R, or S408A mutant G4-MEF2A and HASUMO1 were analyzed as in FIG. 3C.

FIGS. 3F and 3G are immunoblots showing that endogenous MEF2A is sumoylated in neurons. In FIG. 3F, granule neurons in non-depolarizing concentrations of KCl (5 mM) were lysed in the presence or absence of the isopeptidase inhibitor N-ethylmaleimide (NEM) and immunoblotted for MEF2A. Asterisk indicates a form of MEF2A of appropriate size for sumoylated MEF2A. In FIG. 3G, granule neurons are exposed to media containing non-depolarizing (5 mM) or depolarizing (25 mM) concentrations of KCl in the presence of nimodipine (Nim) or its control vehicle (DMSO) were lysed in the presence of NEM and immunoblotted as in FIG. 3F. MEF2A is also acetylated in neurons in a VSCC- and calcineurin-dependent manner.

FIG. 4A is a bar graph. Granule neurons were transfected with wild type, K403R or S408A mutant G4-MEF2A together with the p5G4-E1b-luc and the pRLTK reporter genes. The K403R and S408A mutants of G4-MEF2A had significantly greater transcriptional activity than wild type G4-MEF2A (p<0.05, ANOVA; n=6).

FIG. 4B is a bar graph and a series of immunoblots. Cells coexpressing MEF2A and increasing amounts of MEF2A-SUMO together with a 3-MRE luciferase reporter gene (pMEF2×3-luc) and pRL-TK. MEF2A-SUMO significantly reduced MRE-dependent transcription at all amounts tested (p<0.01, ANOVA; n=5). Lysates were also immunoblotted for MEF2 or ERK1/2.

FIG. 4C is a bar graph. Cerebellar slices were transfected with control vector, MEF2A or MEF2A-SUMO. Dendritic claw number was significantly increased by MEF2A-SUMO (p<0.005, ANOVA; n=53).

FIG. 4D is a bar graph. Cerebellar slices transfected as in FIG. 1C were treated with nimodipine (Nim, 20 μM), cyclosporin A (CsA, 4 μM) or vehicle (DMSO). Dendritic claw number was significantly increased by Nim (p<0.001, t-test; n=119) and CsA (p<0.005, t-test; n=53).

FIG. 4E is a series of immunofluorescent stains and immunoblots, as well as a bar graph. In the upper right panel, lysates of 293T cells transfected with the control or U6/mef2a plasmid together with MEF2A-WT, MEF2A-Res, K403R or S408A mutant of MEF2A-Res and FLAG-14-3-3 were immunoblotted with the indicated antibodies. In the left panel, cerebellar slices transfected with the U6/mef2a plasmid together with MEF2A-Res, or K403R or S408A mutants of MEF2A-Res were analyzed as in FIG. 1E. Scale bar=3 μm. The number of claws was significantly higher in granule neurons that expressed MEF2A-Res but not MEF2A-ResK403R or MEF2A-ResS408A in the presence of MEF2A knockdown when compared to MEF2A knockdown alone (p<0.001, ANOVA; n=133).

FIG. 4F is a bar graph. Cerebellar slices were transfected with the U6/mef2a plasmid together with MEF2A-Res or MEF2A-ResS408ASUMO. The number of claws was significantly higher in neurons expressing MEF2A-Res or MEF2A-ResS408A-SUMO in the presence of MEF2A knockdown when compared to MEF2A knockdown alone (p<0.001, ANOVA; n=105).

FIG. 5A is a picture of a gel. Endogenous MEF2A is associated with the endogenous Nur77 promoter but not nucleolin (control) in granule neurons as determined by chromatin immunoprecipitation analysis.

FIG. 5B is a picture of a RT-PCR gel photograph showing that depolarization induces Nur77 gene expression in neurons in a VSCC- and calcineurin-dependent manner. RNA of granule neurons treated for 1 h in the presence or absence of 25 mM KCl and vehicle (DMSO), nimodipine (Nim), or cyclosporin A (CsA) was subjected to RT-PCR using primers specific to Nur77 or GAPDH.

FIG. 5C is a bar graph. Depolarization of granule neurons significantly induced expression of a luciferase reporter gene controlled by a Nur77 promoter containing wild type (WT) MEF2 response element (MRE) but not of a reporter controlled by a Nur77 promoter containing mutant MRE (MREmut) (p<0.0001, ANOVA; n=6). Treatment with CsA prevented depolarization-induced expression of the WT Nur77-luciferase reporter gene (p<0.0001, ANOVA; n=6), but had no effect on MREmut Nur77-luciferase reporter gene.

FIG. 5D is a bar graph. Nur77-luciferase reporter gene activity was significantly induced in depolarized neurons transfected with the control vector or MEF2A (p<0.005, ANOVA; n=4). Both MEF2A-SUMO and MEF2-EN repressed depolarization-induced Nur77-luciferase reporter activity.

FIG. 5E is a bar graph. Cerebellar slices were transfected with control vector, wild type Nur77 (Nur77-WT) or dominant negative Nur77 (Nur77-DN). The number of claws was significantly increased in neurons expressing Nur77-DN compared to control-transfected neurons or neurons expressing Nur77-WT (p<0.001, ANOVA; n=114).

FIG. 6 is a model of the PIASx-MEF2 sumoylation pathway in the control of postsynaptic dendritic claw differentiation in the cerebellar cortex.

FIG. 7 is a chart showing the Sumoylation-Acetylation Switch (SAS) is a conserved motif in numerous transcription factors and coregulators. Shown are representative conserved pairs of a subset of proteins containing the SAS motif, where Ψ is any large, hydrophobic amino acid. Proteins are listed by human gene name and species. SAS motif containing proteins shown are: ATF6=Activating transcription factor 6 beta; ERR1=Estrogen related receptor alpha; ERR2=Estrogen Related Receptor beta; ERR3=Estrogen relater receptor gamma; ETS1=Erythroblastosis virus E26 oncogene homolog 1; FOXC2=Forkhead box protein C2; GATA-1=GATA binding factor 1; HSF1=Heat shock factor 1; HSF4=Heat shock factor 4; MYB=Myeloblastosis oncogene homolog; NCOA2=Nuclear receptor coactivator 2; NCOR=Nuclear receptor corepressor 1; PPARγ=Peroxisome proliferative activated receptor gamma; SNIP1=SMAD nuclear interacting protein 1; SOX3=SRY-box containing protein 3; SOX8=SRY-box containing protein 8; SOX9=SRY-box containing protein 9; SREBP2=Sterol regulatory element-binding transcription factor 2; THRB1=Thyroid hormone receptor beta-1. Species abbreviations are: Hs=Homo sapiens; Ce=Caenorhabditis elegans; Cg=Cricetulus griseus; Dm=Drosophila melanogaster; Dr=Danio rerio; Gg=Gallus gallus; Mm=Mus musculus; Pf=Platichthys fletus; Xl=Xenopus laevis.

### DETAILED DESCRIPTION OF THE INVENTION

The present invention provides methods for modulating, i.e. increasing or decreasing, neuronal synapse formation by modulating the activity of the transcriptional factor myocyte enhancer factor 2 (MEF2) (e.g., MEF2A), MEF2C, MEF2D, dMEF2, CeMEF2, Activating transcription factor 6 beta (ATF6), Estrogen related receptor alpha (ERR1), Estrogen related receptor beta (ERR2), Estrogen related receptor gamma (ERR3), Erythroblastosis virus E26 oncogene homolog 1 (ETS1), Forkhead box protein C2 (FOXC2), Gata binding factor 1 (GATA-1), Heat shock factor 1 (HSF1), HSF4, MLL3, Myeloblastosis oncogene homolog (MYB), Nuclear receptor coactivator 2 (NCOA2), Nuclear receptor corepressor 1 (NCOR1), Peroxisome proliferative activated receptor gamma (PPARg), SMAD nuclear interacting protein 1 (SNIP1), SRY-box containing protein 3 (SOX3), SOX8, SOX9, Sterol regulatory element-binding transcription factor 2 (SREBP2), or Thyroid hormone receptor beta-1 (THRB1). (See Shalizi et al., 2006, Science 311: 1012-1017, which is incorporated by reference in its entirety.)

The present invention is based on the discovery that a transcription repressor form of myocyte enhancer factor 2A (MEF2A) plays a key role in the morphogenesis of postsynaptic granule neuron dendritic claws in the cerebellar cortex, an essential step in synapse formation. Specifically, sumoylation at Lys403 in the sumoylation-acetylation switch (SAS) peptide of MEF2A promotes dendritic claw differentiation. Activity-dependent calcium signaling induces a calcineurin-mediated dephosphorylation of MEF2A at Ser408 thereby promoting a switch from sumoylation to acetylation at Lys403, and in turn leading to inhibition of dendritic claw differentiation. These findings define a mechanism underlying postsynaptic differentiation that modulate activity-dependent synapse development and plasticity in the brain. Accordingly, the methods and compositions of the invention are useful for modulating, i.e. increasing or decreasing, synapse formation such as dendritic claw differentiation. Methods for screening agents useful for modulating synapse formation are also described herein.

### Screening Assays

Screening methods are carried out to identify compounds that modulate synapse formation by modulating the activity of MEF2A. Useful compounds include any agent that modulates, i.e., increases or reduces Ser408 phosphorylation, Lys403 acetylation, or Lys403 sumoylation within the SAS peptide motif of the MEF2A polypeptide. Other useful compounds are identified by detecting an attenuation of the expression or activity of any of the molecules involved in MEF2A signaling.

A number of methods are available for carrying out such screening assays. According to one approach, candidate compounds are added at varying concentrations to the culture medium of cells expressing a polypeptide containing the SAS peptide motif, such as a MEF2A polypeptide. The level of Ser408 phosphorylation, Lys403 acetylation, or Lys403 sumoylation is then measured, for example, by standard Western blot analysis. The level of gene expression in the presence of the candidate compound is compared to the level measured in a control culture medium lacking the candidate molecule. For example, immunoassays may be used to detect or monitor the level of post-translational modifications, such as phosphorylation levels. Polyclonal or monoclonal antibodies which are capable of binding to the phosphorylated Ser408, for example, may be used in any standard immunoassay format (e.g., ELISA or RIA assay) to measure the level of phosphorylated Ser408. Other techniques that may be used to determine the level of post-translational modifications at the Ser408 and Lys403 residues within the SAS peptide motif include mass spectroscopy, high performance liquid chromatography, spectrophotometric or fluorometric techniques, or combinations thereof.

As a specific example, mammalian cells (e.g., rodent cells) that express a nucleic acid encoding MEF2A containing the SAS peptide motif are cultured in the presence of a candidate compound (e.g., a peptide, polypeptide, synthetic organic molecule, naturally occurring organic molecule, nucleic acid molecule, or component thereof). Cells may either endogenously express MEF2A or may alternatively be genetically engineered by any standard technique known in the art (e.g., transfection and viral infection) to overexpress MEF2A. The level of Ser408 phosphorylation is measured in these cells by means of Western blot analysis and subsequently compared to the level of expression of Ser408 phosphorylation in control cells that have not been contacted by the candidate compound. A compound which modulates the level of Ser408 phosphorylation is considered useful in the invention.

Alternatively, the screening methods of the invention may be used to identify candidate compounds that modulate synapse formation as a result of a modulation in MEF2A activity by modulating Lys403 acetylation levels by at least 10%, 20%, 30%, 40%, 50%, 60%, 70%, 80%, 90%, 95%, or 100% relative to an untreated control. As an example, a candidate compound may be tested for its ability to increase Lys403 acetylation within the SAS peptide motif of MEF2A in cells that naturally express MEF2A, after transfection with cDNA for MEF2A, or in cell-free solutions containing MEF2A. The effect of a candidate compound on the binding or activation of MEF2A can be tested by radioactive and non-radioactive binding assays, competition assays, and receptor signaling assays.

Given its ability to modulate the biological activity of MEF2A, such a molecule may be used, for example, as a therapeutic agent to modulate synapse formation, or alternatively, to alleviate one or more symptoms associated with CNS injury or a psychiatric or neurologic disorder. As a specific example, a candidate compound may be contacted with two proteins, the first protein being a polypeptide substantially identical to MEF2A (i.e. a protein that contains a SAS peptide motif) and the second protein being a serine kinase (i.e., a protein that binds and phosphorylates MEF2A at Ser408 under conditions that allow binding and phosphorylation). According to this particular screening method, the interaction between these two proteins is measured following the addition of a candidate compound. A decrease in the binding of the first protein to the second protein following the addition of the candidate compound (relative to such binding in the absence of the compound) identifies the candidate compound as having the ability to inhibit the interaction between the two proteins, and thereby having the ability to reduce Ser408 phosphorylation. This compound would therefore be useful to reduce synapse formation. The screening assay of the invention may be carried out, for example, in a cell-free system or using a yeast two-hybrid system. If desired, one of the proteins or the candidate compound may be immobilized on a support as described above or may have a detectable group.

Alternatively, or in addition, candidate compounds may be screened for those which specifically bind to Ser408 phosphorylated MEF2A, or alternatively Lys403 sumoylated or acetylated MEF2A, and thereby modulate synapse formation. The efficacy of such a candidate compound is dependent upon its ability to interact with MEF2A. Such an interaction can be readily assayed using any number of standard binding techniques and functional assays. For example, a candidate compound may be tested in vitro for interaction and binding with MEF2A and its ability to modulate synapse formation may be assayed by any standard assays (e.g., those described herein).

For example, a candidate compound that binds specifically to the acetylated Lys403 MEF2A may be identified using a chromatography-based technique. For example, a recombinant SAS peptide motif with an acetylated Lys403 residue may be purified by standard techniques from cells engineered to express this polypeptide (e.g., those described above) and may be immobilized on a column. A solution of candidate compounds is then passed through the column, and a compound specific for the acetylated Lys403 SAS peptide is identified on the basis of its ability to bind to acetylated Lys403 and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography).

Screening for new inhibitors and optimization of lead compounds may be assessed, for example, by assessing their ability to modulate MEF2A activity using standard techniques. In addition, these candidate compounds may be tested for their ability to function as modulators of synapse formation (e.g., as described herein). Compounds which are identified as binding to MEF2A with an affinity constant less than or equal to 10 mM are considered particularly useful in the invention.

Antibodies are used to measure PIASx/MEF2A association by resonance energy transfer such as Fluorescence Resonance Energy Transfer (FRET) or Bioluminescence Resonance Energy Transfer (BRET). For example, a FRET assay is carried out as follows. Each antibody is coupled to a different fluorochrome. One fluorochrome emits at a higher energy than the excitation energy of the second fluorochrome. These antibody-fluorochrome conjugates are then applied to freshly isolated neural cells (not fixed) and exposed to a light source that activates fluorochrome 1 but not 2. Fluorochrome 2 absorbs light reemitted from fluorochrome 1. The amount of energy transferred is a function of distance. This assay measures a change in the distance between the site occupied by the first antibody and the second antibody, and thus indicates PIASx/MEF2A association.

### A High-Throughput Screen for Modifiers of SAS Motif Function

A rapid screen for compounds useful for modulating synapse formation is carried out using either biomolecular enzymatic complementation (BiEC) or biomolecular fluorescence complementation (BiFC) assays (e.g., Rossi et al., Meth. Enzymol. 2000:328:231-51 or Hu and Kerppola, Nat. Biotechnol, 2003:21:539-45). In the former example, a gene fusion of the MEF2A SAS motif containing peptide fused to a N- or C-terminal fragment of beta-galactosidase is expressed in mammalian cells, preferably neural cells, together with a gene fusion of the SUMO protein fused to the complementary (N- or C-terminal) fragment of beta-galactosidase. Cells are then contacted with candidate compounds for modulating synapse formation, such as small molecules or shRNAs, and changes in the enzymatic activity of beta-galactosidase is measured. Candidate compounds that increase sumoylation of the SAS motif increase beta-galactosidase complementation and enzymatic activity, and are thus compounds that are useful to increase synapse formation. Candidate compounds that reduce sumoylation of the SAS motif reduce beta-galactosidase complementation and enzymatic, and are thus compounds that are useful to reduce synapse formation.

For BiFC, a gene fusion of the MEF2A SAS motif-containing peptide fused to a N- or C-terminal fragment of GFP is expressed in mammalian cells, preferably neural cells, together with a gene fusion of the SUMO protein fused to the complementary (N- or C-terminal) fragment of GFP. Cells are then contacted with candidate compounds for modulating synapse formation, such as small molecules or shRNAs, and changes in the fluorescence activity of GFP is measured. Candidate compounds that increase sumoylation of the SAS motif increase GFP fluorescence, and are thus compounds that are useful to increase synapse formation. Candidate compounds that reduce sumoylation of the SAS motif would reduce GFP complementation and fluorescence, and are thus compounds that are useful to reduce synapse formation. The methods described above represent rapid, cell-based assays for screening small-molecule libraries or shRNA libraries for modulators of synapse formation.

Therapeutic Agents

A compound that is useful for modulating synapse formation (e.g., by promoting dendritic differentiation) is one having the ability to increase or reduce the level of Serin408 (Ser408) phosphorylation of the SAS peptide motif in MEF2A. Other useful compounds include those that increase or reduce the level of sumoylation or acetylation at lysine 403 (Lys403) in the SAS peptide motif. A compound that increases synaptic function is a one that increases phosphorylation of Ser408, increases sumoylation at Lys403, or reduces acetylation at Lys403Exemplary agents that increase the level of Ser408 phosphorylation are phosphatase inhibitors, such as cyclosporin A and FK506. Conversely, a compound that reduces synaptic function is a compound that reduces phosphorylation of Ser408, reduces sumoylation at Lys403, or increases acetylation at Lys403. An agent that reduces Ser408 phosphorylation in the SAS peptide motif of MEF2A, thereby reducing synapse formation, is a kinase inhibitor or an agent that increases the activity of a phosphatase. Agents that reduce the level of acetylation, thereby increasing synapse formation, include nimodipine, curcumin and its derivatives, HAT inhibitors, and VSCC or calcineurin inhibitors such as CsA. Agents that increase the level of acetylation thereby reducing synapse formation include agents that reduce the expression or activity level of a histone deacetylase (HDAC) (e.g., class I HDAC, class II HDAC, and class III HDAC), trichostatin A, suberoylanilide hydroxamic acid (SAHA), pyroxamide, apicidin, depudecin, depsipeptide, oxamflatin, CI-994 (N-acetyl dinaline), m-Carboxy cinnamic acid bishydroxamic acid (CBHA), scriptaid, trapoxin, TPX-HA analogue (CHAP), and sirtinol. Other exemplary agents are agents that increase the expression or activity level of a histone acetyltransferase. An agent that reduces the level of sumoylation includes, for example, an agent that reduces the expression or activity level of SUMO activating enzymes, Ubc9, or SUMO E3 ligase. Optionally, the agent is a SUMO-removing isopeptidase. Agents that increase the level of sumoylation in the cell, thereby increasing synapse formation, include agents that increase the expression of the SUMO-conjugating enzyme Ubc9 or the expression of a SUMO E3 ligase. Other agents that increase the level of sumoylation also reduce the level of acetylation at Lys403 and include agents that reduce the expression or activity level of a histone acetyl transferase enzyme, such as curcumin and its derivatives as well as HAT inhibitors. Other agents that increase the level of sumoylation at Lys403 include the isopeptidase inhibitor N-ethylmaleimide, or nimodipine or similar voltage-sensistive calcium channel (VSCC) or calcineurin inhibitors, including cyclosporin A (CsA).

The level of post-translational modification at an amino acid residue (including Ser408 phosphorylation, Lys403 acetylation, or Lys403 sumoylation) is determined by any standard method in the art, including those described herein. Synapse formation modulators include polypeptides, polynucleotides, small molecule antagonists, and siRNA.

For example, the synapse formation modulator is a dominant negative protein or a nucleic acid encoding a dominant negative protein that interferes with the biological activity of MEF2A. A dominant negative protein is any amino acid molecule having a sequence that has at least 50%, 70%, 80%, 90%, 95%, or even 99% sequence identity to at least 10, 20, 35, 50, 100, or more than 150 amino acids of the wild type protein to which the dominant negative protein corresponds. For example, a dominant-negative MEF2A has mutation within the SAS peptide motif such that it can no longer be phosphorylated at the Ser408 position.

The dominant negative protein may be administered as an expression vector. The expression vector may be a non-viral vector or a viral vector (e.g., recombinant retrovirus, recombinant lentivirus, recombinant adeno-associated virus, or a recombinant adenoviral vector). Alternatively, the dominant negative protein may be directly administered as a recombinant protein systemically or to the infected area using, for example, microinjection techniques.

The synapse formation modulator is an antisense molecule, an RNA interference (siRNA) molecule such as hpRNA, or a small molecule antagonist that targets the activity of MEF2A, by modulating the phosphorylation level of Ser 408 or by modulating the acetylation or the sumoylation level of Lys403. By the term “siRNA” is meant a double stranded RNA molecule which degrades a target mRNA or prevents translation of a target mRNA. Standard techniques of introducing siRNA into a cell are used, including those in which DNA is a template from which an siRNA RNA is transcribed. The siRNA includes a sense SAS peptide motif nucleic acid sequence, an anti-sense SAS peptide motif nucleic acid sequence or both. Optionally, the siRNA is constructed such that a single transcript has both the sense and complementary antisense sequences from the target gene, e.g., a hairpin. Binding of the siRNA to a SAS peptide motif transcript in the target cell results in modulation of the level of Ser408 phosphorylation, Lys403 acetylation, or Lys403 sumoylation in the SAS peptide motif of MEF2A. The length of the oligonucleotide is at least 10 nucleotides and may be as long as the naturally-occurring SAS peptide motif transcript, or even the MEF2A transcript. Preferably, the oligonucleotide is 19-25 nucleotides in length. Most preferably, the oligonucleotide is less than 75, 50, 25 nucleotides in length.

Small molecules includes, but are not limited to, peptides, peptidomimetics (e.g., peptoids), amino acids, amino acid analogs, polynucleotides, polynucleotide analogs, nucleotides, nucleotide analogs, organic and inorganic compounds (including heterorganic and organomettallic compounds) having a molecular weight less than about 5,000 grams per mole, organic or inorganic compounds having a molecular weight less than about 2,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The preferred dose of the synapse formation modulator is a biologically active dose. A biologically active dose is a dose that will increase or reduce synapse formation. The levels of Ser408 phosphorylation and the levels of Lys403 sumoylation or acetylation may be determined by any method known in the art, including, for example, Western blot analysis, immunohistochemistry, ELISA, and Northern Blot analysis. Alternatively, the biological activity of MEF2A or any of the molecules that are involved in MEF2A signaling may be determined. The biological activity of MEF2A is determined according to its ability to increase or reduce synapse formation.

Optionally, the subject is administered one or more additional therapeutic regiments in addition to the synapse formation modulator. The additional therapeutic regimens may be administered prior to, concomitantly, or subsequent to administration of the synapse formation modulator. For example, the synapse formation modulator and the additional agent are administered in separate formulations within at least 1, 2, 4, 6, 10, 12, 18, or more than 24 hours apart. Optionally, the additional agent is formulated together with the synapse formation modulator. When the additional agent is present in a different composition, different routes of administration may be used. The agent is administered at doses known to be effective for such agent for modulating synapse formation.

Concentrations of the synapse formation modulator and the additional agent depends upon different factors, including means of administration, target site, physiological state of the mammal, and other medication administered. Thus treatment dosages may be titrated to optimize safety and efficacy and is within the skill of an artisan. Determination of the proper dosage and administration regime for a particular situation is within the skill of the art.

Treatment is efficacious if the treatment leads to clinical benefit such as, a reduction of the symptoms in the subject. When treatment is applied prophylactically, the treatment retards or prevents symptoms from occurring. Efficacy may be determined using any known method for diagnosing or treating the disorder being treated.

### Administration of Compounds

The invention includes administering to a subject a composition that includes a compound that modulates synapse formation (referred to herein as an “synapse formation modulator” or “therapeutic compound”).

An effective amount of a therapeutic compound is preferably from about 0.1 mg/kg to about 150 mg/kg. Effective doses vary, as recognized by those skilled in the art, depending on route of administration, excipient usage, and coadministration with other therapeutic treatments including use of other therapeutic agents for treating, preventing or alleviating a symptom of the disorder being treated. A therapeutic regimen is carried out by identifying a mammal, e.g., a human patient suffering from CNS injury, psychiatric disorder or neurologic disorder, using standard methods.

The pharmaceutical compound is administered to such an individual using methods known in the art. Preferably, the compound is administered orally, rectally, nasally, topically or parenterally, e.g., subcutaneously, intraperitoneally, intramuscularly, and intravenously. The compound is administered prophylactically, or after the detection of a psychiatric disorder or neurologic disorder. The compound is optionally formulated as a component of a cocktail of therapeutic drugs. Examples of formulations suitable for parenteral administration include aqueous solutions of the active agent in an isotonic saline solution, a 5% glucose solution, or another standard pharmaceutically acceptable excipient. Standard solubilizing agents such as PVP or cyclodextrins are also utilized as pharmaceutical excipients for delivery of the therapeutic compounds.

The therapeutic compounds described herein are formulated into compositions for other routes of administration utilizing conventional methods. For example, the synapse formation modulator is formulated in a capsule or a tablet for oral administration. Capsules may contain any standard pharmaceutically acceptable materials such as gelatin or cellulose. Tablets may be formulated in accordance with conventional procedures by compressing mixtures of a therapeutic compound with a solid carrier and a lubricant. Examples of solid carriers include starch and sugar bentonite. The compound is administered in the form of a hard shell tablet or a capsule containing a binder, e.g., lactose or mannitol, a conventional filler, and a tableting agent. Other formulations include an ointment, suppository, paste, spray, patch, cream, gel, resorbable sponge, or foam. Such formulations are produced using methods well known in the art.

Where the therapeutic compound is a nucleic acid encoding a protein, the Therapeutic nucleic acid is administered in vivo to promote expression of its encoded protein, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide which is known to enter the nucleus (See, e.g., Joliot, et al., 1991. Proc Natl Acad Sci USA 88:1864-1868). A nucleic acid therapeutic is introduced intracellularly and incorporated within host cell DNA or remain episomal.

Optionally, standard gene therapy vectors are used for local administration of DNA to modulate the level of sumoylation, phosphorylation, or acetylation in the cell. Exogenous DNA encoding agents that modulate the level of sumoylation, phosphorylation, or acetylation is administered to increase synapse formation. Alternatively, the expression of agents that modulate the level of sumoylation, phosphorylation, or acetylation in the cell is reduced or knocked-down using small interfering RNA (siRNA), microRNA (miRNA), antisense, hairpin RNA, or RNAi strategies. Alternatively, any mechanism that interferes with transcription or translation is used to knockdown the expression of an agent that modulates the level of sumoylation, phosphorylation, or acetylation in the cell.

For local administration of DNA, standard gene therapy vectors are used. Such vectors include viral vectors, including those derived from replication-defective hepatitis viruses (e.g., HBV and HCV), retroviruses (see, e.g., WO 89/07136; Rosenberg et al., 1990, N. Eng. J. Med. 323(9):570-578), adenovirus (see, e.g., Morsey et al., 1993, J. Cell. Biochem., Supp. 17E), adeno-associated virus (Kotin et al., 1990, Proc. Natl. Acad. Sci. USA 87:2211-2215,), replication defective herpes simplex viruses (HSV; Lu et al., 1992, Abstract, page 66, Abstracts of the Meeting on Gene Therapy, September 22-26, Cold Spring Harbor Laboratory, Cold Spring Harbor, N.Y.), and any modified versions of these vectors. The invention may utilize any other delivery system which accomplishes in vivo transfer of nucleic acids into eukaryotic cells. For example, the nucleic acids may be packaged into liposomes, e.g., cationic liposomes (Lipofectin), receptor-mediated delivery systems, non-viral nucleic acid-based vectors, erythrocyte ghosts, or microspheres (e.g., microparticles; see, e.g., U.S. Pat. No. 4,789,734; U.S. Pat. No. 4,925,673; U.S. Pat. No. 3,625,214; Gregoriadis, 1979, Drug Carriers in Biology and Medicine, pp. 287-341 (Academic Press,). Naked DNA may also be administered.

DNA for gene therapy can be administered to patients parenterally, e.g., intravenously, subcutaneously, intramuscularly, and intraperitoneally. DNA or an inducing agent is administered in a pharmaceutically acceptable carrier, i.e., a biologically compatible vehicle which is suitable for administration to an animal e.g., physiological saline. A therapeutically effective amount is an amount which is capable of producing a medically desirable result, e.g., a modulation in synapse formation in a treated animal. Such an amount can be determined by one of ordinary skill in the art. As is well known in the medical arts, dosage for any given patient depends upon many factors, including the patient's size, body surface area, age, the particular compound to be administered, sex, time and route of administration, general health, and other drugs being administered concurrently. Dosages may vary, but a preferred dosage for intravenous administration of DNA is approximately 10⁶ to 10²² copies of the DNA molecule.

Typically, plasmids are administered to a mammal in an amount of about 1 nanogram to about 5000 micrograms of DNA. Desirably, compositions contain about 5 nanograms to 1000 micrograms of DNA, 10 nanograms to 800 micrograms of DNA, 0.1 micrograms to 500 micrograms of DNA, 1 microgram to 350 micrograms of DNA, 25 micrograms to 250 micrograms of DNA, or 100 micrograms to 200 micrograms of DNA. Alternatively, administration of recombinant adenoviral vectors encoding the agent into a mammal may be administered at a concentration of at least 10^{5, 10}⁶, 10⁷, 10⁸, 10⁹, 10¹⁰, or 10¹¹ plaque forming unit (pfu).

Gene products are administered to the patient intravenously in a pharmaceutically acceptable carrier such as physiological saline. Standard methods for intracellular delivery of peptides can be used, e.g. packaged in liposomes. Such methods are well known to those of ordinary skill in the art. It is expected that an intravenous dosage of approximately 1 to 100 moles of the polypeptide of the invention would be administered per kg of body weight per day. The compositions of the invention are useful for parenteral administration, such as intravenous, subcutaneous, intramuscular, and intraperitoneal.

Synapse formation modulators are effective upon direct contact of the compound with the affected tissue or may alternatively be administered systemically (e.g., intravenously, rectally or orally). The modulator may be administered intravenously or intrathecally (i.e., by direct infusion into the cerebrospinal fluid in the brain). For local administration, a compound-impregnated wafer or resorbable sponge is placed in direct contact with CNS tissue. The compound or mixture of compounds is slowly released in vivo by diffusion of the drug from the wafer and erosion of the polymer matrix. Alternatively, the compound is infused into the brain or cerebrospinal fluid using standard methods. For example, a burr hole ring with a catheter for use as an injection port is positioned to engage the skull at a burr hole drilled into the skull. A fluid reservoir connected to the catheter is accessed by a needle or stylet inserted through a septum positioned over the top of the burr hole ring. A catheter assembly (described, for example, in U.S. Pat. No. 5,954,687) provides a fluid flow path suitable for the transfer of fluids to or from selected location at, near or within the brain to allow administration of the drug over a period of time.

One in the art will understand that the patients treated according to the invention may have been subjected to the tests to diagnose a subject as having a psychiatric disorder or neurologic disorder may have been identified, without examination, as one at high risk due to the presence of one or more risk factors (e.g., genetic predisposition). Reduction of psychiatric disorder or neurologic disorder symptoms or damage may also include, but are not limited to, alleviation of symptoms, diminishment of extent of disease, stabilized (i.e., not worsening) state of disease, delay or slowing of disease progression, and amelioration or palliation of the disease state. Treatment may occur at home with close supervision by the health care provider, or may occur in a health care facility.

This invention is based in part on the experiments described in the following examples. These examples are provided to illustrate the invention and should not be construed as limiting.

### Example 1

### MEF2A is Essential in Post-Synaptic Dendritic Claw Morphogenesis

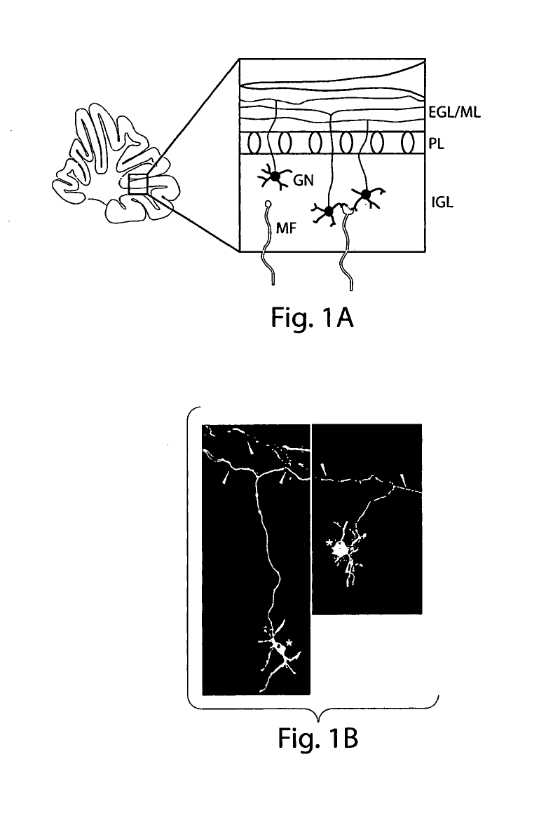

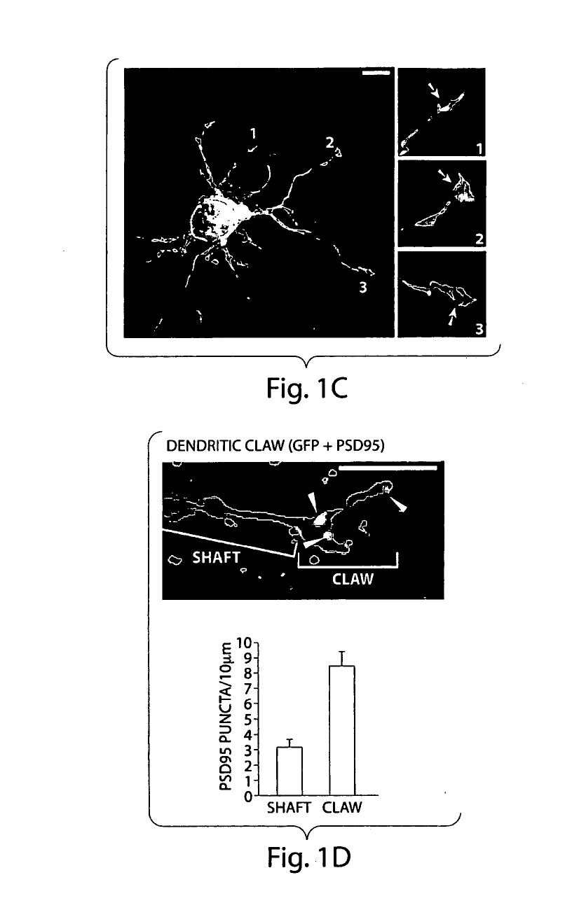

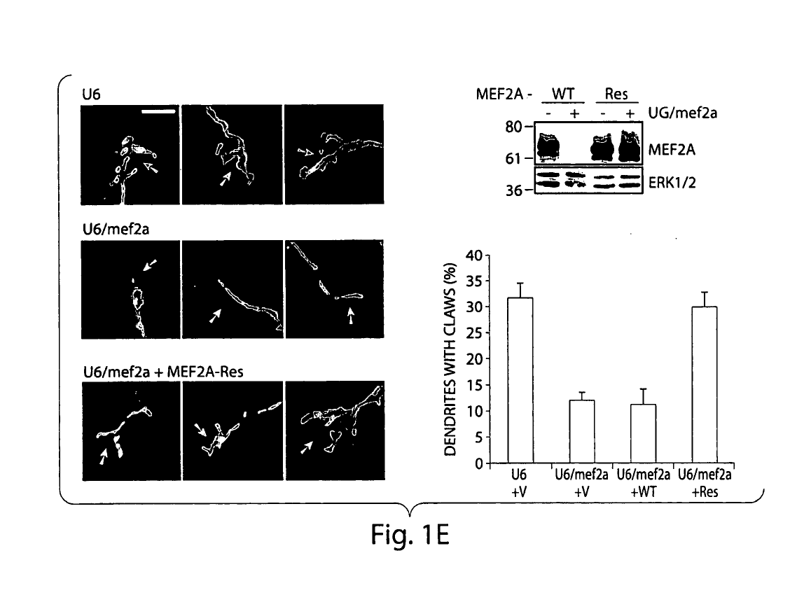

The MEF2 family of transcription factors are highly expressed in the brain as neurons undergo dendritic maturation and synapse formation. MEF2A is especially abundant in granule neurons of the cerebellar cortex throughout the period of synaptogenesis. The role that MEF2A plays in synaptic dendritic development in the cerebellar cortex was determined. During cerebellar development, granule neuron dendritic morphogenesis culminates in the differentiation of dendritic claws upon which mossy fiber terminals and Golgi neuron axons synapse. To visualize granule neurons undergoing postsynaptic differentiation, organotypic cerebellar slices prepared from postnatal day 9 (P9) rat pups were transfected with an expression plasmid encoding green fluorescent protein (GFP). Transfected granule neurons in the internal granular layer (IGL) had the typical small cell body with associated parallel axonal fibers and few dendrites (FIGS. 1A and 1B). Many dendrites harbored structures with the appearance of dendritic claws that were identified based on classic descriptions as: (i) located at the end of a dendrite, (ii) having cup- or sickle-like appearance, and (iii) displaying undulating or serrated inner surfaces (FIGS. 1C and 1D). Dendritic claws showed punctuate expression of the postsynaptic protein PSD95 (FIG. 1D). PSD95 puncta density was greater in the claw region than in the shaft of dendrites (FIG. 1D). Thus, granule neuron dendritic claws in cerebellar slices represent sites of postsynaptic differentiation. The effect of MEF2A knockdown on granule neuron dendritic morphogenesis was next determined. Cerebellar slices were transfected with the U6/mef2a plasmid that encodes MEF2A hairpin RNAs (MEF2AhpRNA) or the control U6 plasmid together with a GFP expression plasmid. The MEF2AhpRNA-expressing granule neurons had 60% fewer dendritic claws than control U6-transfected neurons, and their dendrites displayed tapered or bulbous tips instead of claws (FIG. 1E). In these dendrites, PSD95 puncta density was low in the tip region and no greater than in the shaft. The MEF2AhpRNA-induced dendritic claw phenotype was not due to a reduction in dendritic growth. These results indicate that MEF2A plays a key role in the morphogenesis of dendritic claws in the cerebellar cortex.

To exclude the possibility that the MEF2A knockdown-induced dendritic phenotype is the result of off-target effects of RNAi, a rescue experiment was performed. MEF2A RNAi induced the effective knockdown of MEF2A protein encoded by wild type MEF2A cDNA but failed to reduce the expression of MEF2A encoded by an RNAi-resistant cDNA (MEF2A-Res) (FIG. 1E). In cerebellar slices, MEF2A-Res but not MEF2A-WT reversed the MEF2AhpRNA induced dendritic claw phenotype (FIG. 1E). Expression of MEF2A-Res induced dendritic claws of similar number, morphological appearance, and PSD95 density as those in control U6-transfected neurons (FIGS. 1D, 1E). These experiments indicate that the MEF2AhpRNA induced-dendritic claw phenotype is the result of the specific knockdown of MEF2A.

### Example 2

### MEF2A is Required for Postsynaptic Dendritic Claw Differentiation in vivo

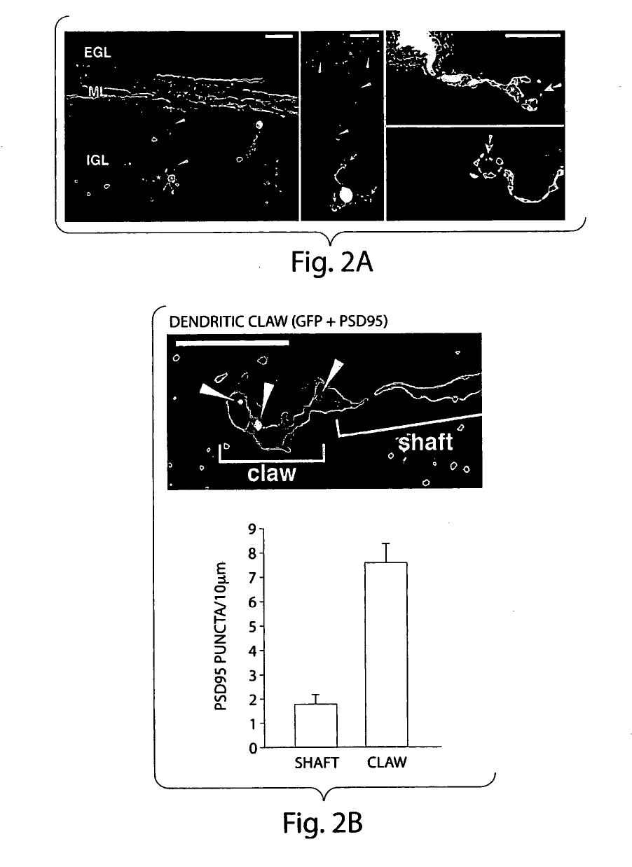

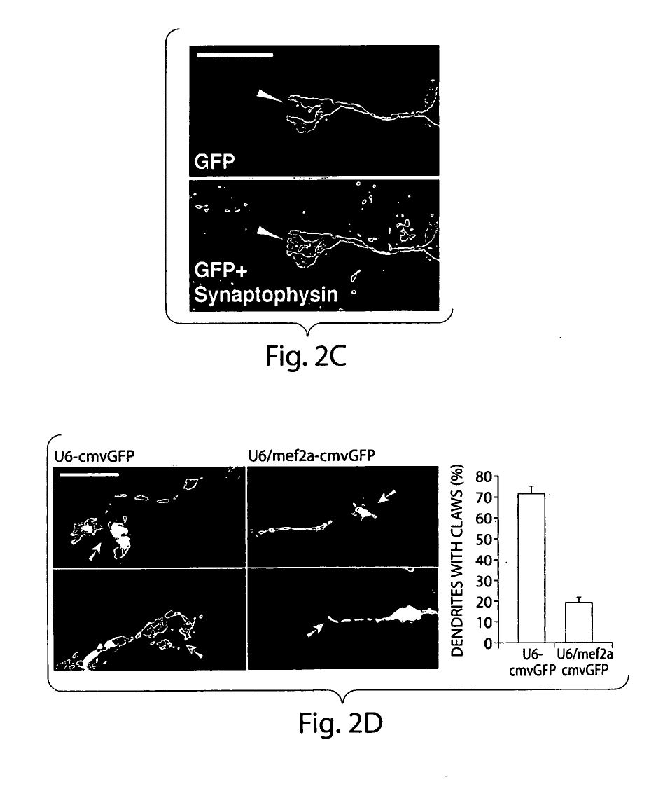

To establish MEF2A function in dendritic claw development in vivo, MEF2A knockdown was induced in the postnatal cerebellum using electroporation-mediated gene transfer. A control U6 or U6/mef2a plasmid that also encoded GFP was injected into the cerebellar cortex of P3 rat pups, and dendritic claws were identified in the cerebellum of these animals at P12. Granule neurons in control-transfected cerebella had PSD95-positive postsynaptic dendritic claws at the tips of their dendrites (FIGS. 2A and B). In addition, expression of the presynaptic protein synaptophysin was found juxtaposed to the surface of approximately 80% of dendritic claws (FIG. 2C). As in cerebellar slices, MEF2A knockdown reduced the number of dendritic claws in the cerebellum in vivo (FIG. 2D). These findings indicate a physiological, cellautonomous function for MEF2A in the morphogenesis of dendritic claws in the developing cerebellar cortex.

### Example 3

### MEF2A Ser408 Dephosphorylation Promotes a Sumoylation to Acetylation Switch at MEF2A Lys403

Calcium signaling strongly influences the activity of MEF2s. Calcium entry through voltage-sensitive calcium channels (VSCCs) triggers MEF2 phosphorylation at distinct sites, and calcineurin-mediated dephosphorylation at undetermined sites, both leading to enhanced MEF2-dependent transcription. Calcineurin has emerged as a critical regulator of dendritic spine morphology in hippocampal neurons. Calcineurin may therefore control postsynaptic dendritic differentiation via a MEF2-regulated transcriptional mechanism.

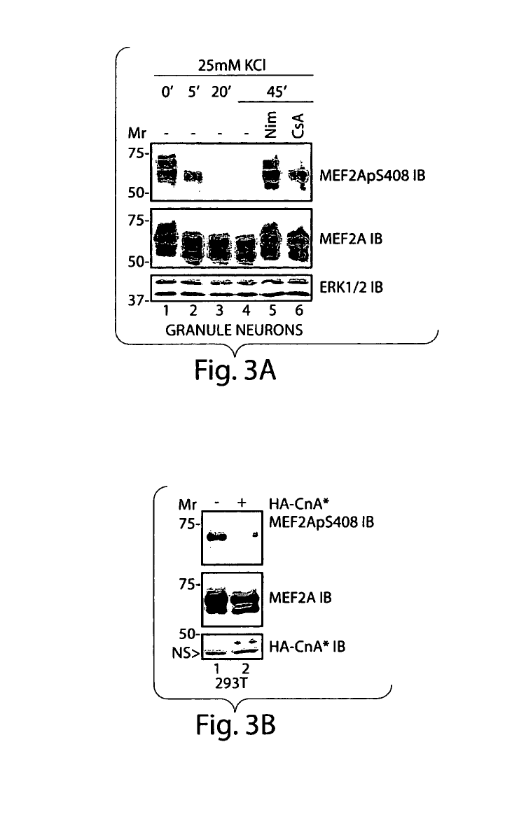

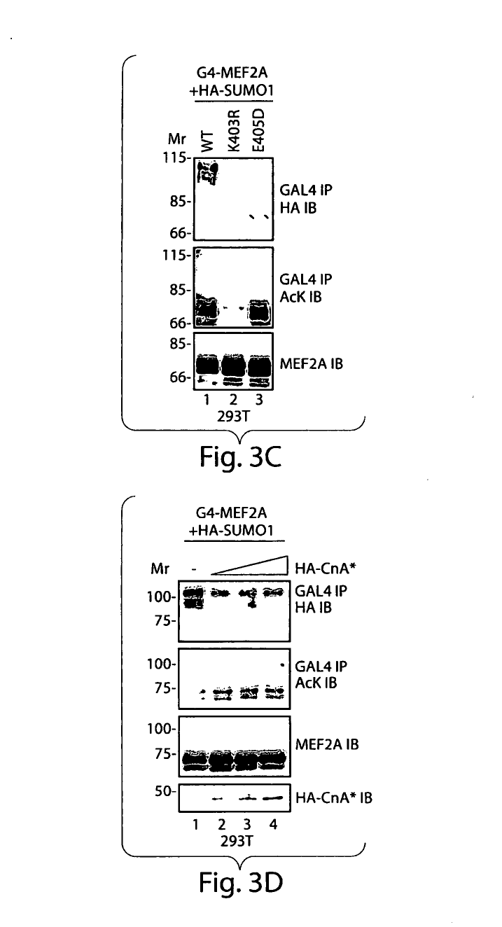

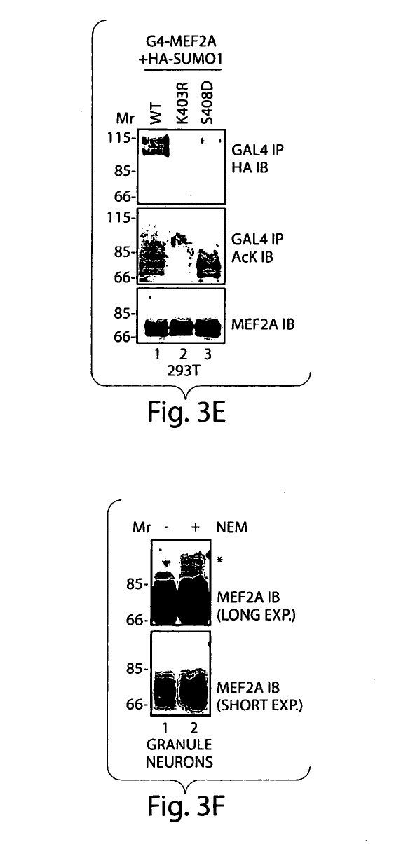

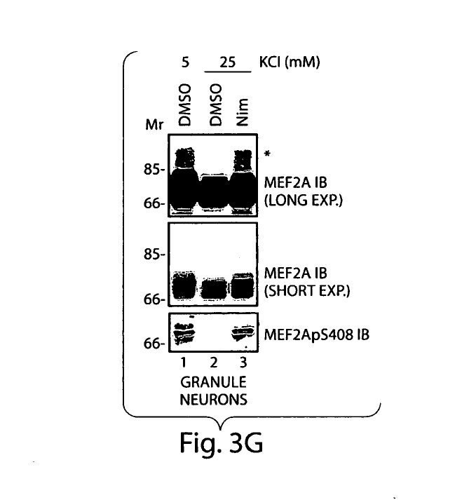

The site of calcineurin-mediated dephosphorylation of MEF2A was determined. Because calcineurin stimulates MEF2-dependent transcription, calcineurin may induce the dephosphorylation of MEF2A at Ser408, whose phosphorylation inhibits MEF2-dependent transcription. Using antibodies that recognize MEF2A when phosphorylated on Ser408, endogenous MEF2A was found to be phosphorylated on Ser408 in neurons (FIG. 3A). Upon membrane depolarization of neurons, MEF2A underwent rapid and robust dephosphorylation at Ser408, an effect that was blocked in neurons treated with nimodipine, an inhibitor of L-type VSCCs, or cyclosporin A (CsA), an inhibitor of calcineurin (FIG. 3A). In 293T cells, MEF2A was constitutively phosphorylated at Ser408, and coexpression of activated calcineurin induced dephosphorylation of MEF2A at this site (FIG. 3B). These results indicate that calcineurin mediates activity-induced dephosphorylation of MEF2A at Ser408 in neurons, despite the fact that Ser408 lies near a conserved SUMO acceptor site centered at Lys403 within a domain of MEF2A that represses transcription. Sumoylation of transcription factors typically induces transcriptional repression. MEF2 proteins function as activators or repressors of transcription in a signal-dependent manner. Whether Ser408 dephosphorylation regulates MEF2A sumoylation at Lys403 and MEF2's transcriptional repression function was next determined. First, MEF2A Lys403 was shown to be modified by sumoylation in vitro and in cells (FIG. 3C). MEF2A was also acetylated in cells in a Lys403-dependent manner (FIG. 3C). To determine how dephosphorylation of Ser408 might regulate the Lys403 modifications, the MEF2A transactivation domain fused to the DNA binding domain of GAL4 (G4-MEF2A) was expressed together with a constitutively active form of calcineurin. Activated calcineurin inhibited sumoylation and enhanced acetylation of MEF2A in cells (FIG. 3D). A G4-MEF2A mutant in which Ser408 was replaced with alanine (G4-MEF2AS408A) had reduced sumoylation and enhanced acetylation when compared to G4-MEF2A (FIG. 3E). Expression of the SUMO E2 ligase Ubc9 in cells increased sumoylation and inhibited the acetylation of G4-MEF2A, but not of G4-MEF2AS408A. These results indicate that the calcineurin-induced dephosphorylation of MEF2A at Ser408 promotes a sumoylation to acetylation switch at Lys403.

In granule neurons, endogenous sumoylated MEF2A was detected as a N-ethylmaleimide (NEM)-sensitive MEF2 immunoreactive band of appropriate molecular size by immunoblotting with antibodies to MEF2A (FIG. 3F). Membrane depolarization of neurons led to an almost complete reduction of sumoylated MEF2A, an effect that tightly correlated with Ser408 dephosphorylation (FIG. 3G). Endogenous MEF2A was acetylated in depolarized neurons. Incubation of depolarized neurons with the VSCC inhibitor nimodipine or the calcineurin inhibitor CsA increased sumoylation and decreased acetylation of endogenous MEF2A (FIG. 3G).

### Example 4

### A Calcium-Regulated Lys403-Sumoylated Transcriptional Repressor form of MEF2A Promotes Dendritic Claw Differentiation

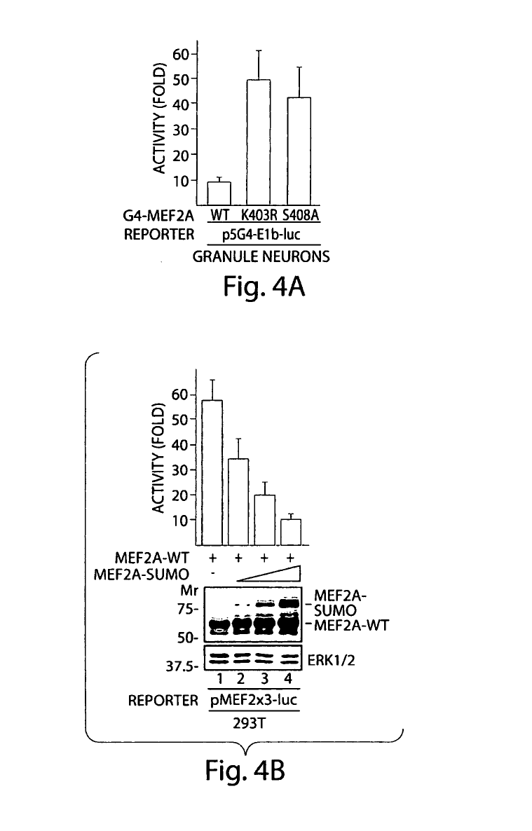

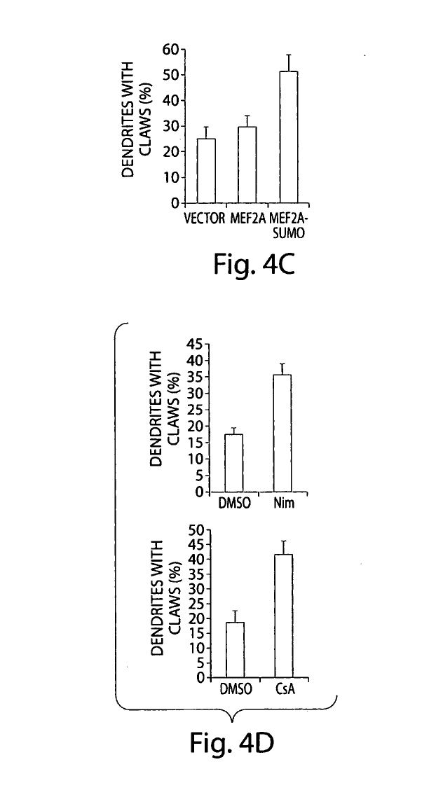

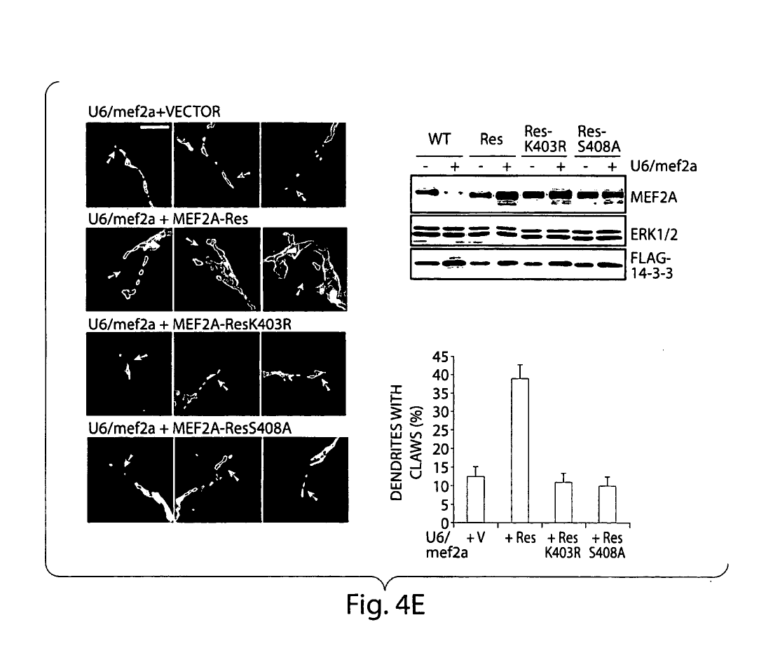

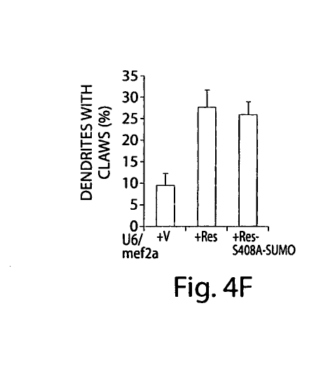

The consequences of Ser408 dephosphorylation-induced Lys403 modifications of MEF2A on transcription were next assessed. Replacement of Ser408 with alanine or Lys403 with arginine in G4-MEF2A similarly enhanced transcription in neurons or 293T cells (FIG. 4A). The S408A and K403R mutants of MEF2A were both deficient in sumoylation, while the S408A mutant enhanced MEF2A acetylation (FIG. 3E). In view of these results, the phenocopy of the S408A and K403R mutants in the reporter assay indicate that sumoylation is the critical modification of Lys403, leading to repression of transcription and that acetylation of Lys403 serves to prevent sumoylation of MEF2A. Fusion of a SUMO moiety to transcription factors mimics the effect of SUMO that is covalently linked to proteins on the native lysine. A MEF2A-SUMO fusion protein potently inhibited the ability of co-expressed wild type MEF2A to induce a MEF2-responsive (MREdependent) reporter gene in cells (FIG. 4B). Sumoylation did not appear to alter MEF2A's subnuclear localization or stability. These findings indicate that Lys403-sumoylated MEF2A represses transcription, and that Ser408 dephosphorylation inhibits Lys403 sumoylation and thereby derepresses MEF2A-induced transcription.

To characterize the role of the calcium-MEF2A signaling pathway in dendritic claw morphogenesis in the cerebellar cortex, the effect of MEF2A sumoylation on dendritic claw differentiation was tested. Expression of MEF2A-SUMO increased the number of dendritic claws compared to MEF2A-expressing or control transfected neurons (FIG. 4C), indicating that a transcriptional repressor form of MEF2A stimulates dendritic claw differentiation. Furthermore, expression of a protein in which the MADS/MEF2 domains were fused to the transcriptional repressor Engrailed (MEF2-EN), which potently repressed MRE-dependent transcription, led to an increase in the number of dendritic claws in cerebellar slices.

The effect of the endogenous calcium-induced cascade of modifications at Ser408 and Lys403 of MEF2A on dendritic claw differentiation was next determined. Incubation of cerebellar slices with nimodipine or CsA increased dendritic claw number (FIG. 4D), suggesting that VSCC or calcineurin activation inhibits dendritic claw development. The ability of a S408A or K403R mutant of MEF2A-Res to rescue the MEF2AhpRNA-induced dendritic claw phenotype in cerebellar slices was next tested. The S408A mutant mimicked calcineurin-induced dephosphorylation of MEF2A Ser408, while both S408A and K403R mutants of MEF2A were deficient in sumoylation and transcriptional repression (FIGS. 3 and 4A). In contrast to MEF2A-Res, neither mutant of MEF2A-Res reversed MEF2AhpRNA-inhibition of dendritic claw morphogenesis (FIG. 4E). Fusion of SUMO with the S408A mutant of MEF2A-Res protein conferred this protein with the ability to induce dendritic claw differentiation in the presence of MEF2A knockdown (FIG. 4F). Thus, the rescue experiments suggest that an endogenously-sumoylated transcriptional repressor form of MEF2A promotes dendritic claw differentiation. Together, our results also suggest that the calcium-induced Ser408 dephosphorylation and consequent inhibition of Lys403 sumoylation of MEF2A suppress dendritic claw morphogenesis.

### Example 5

### Nur77 Repression by MEF2A-SUMO Contributes to Dendritic Claw Morphogenesis

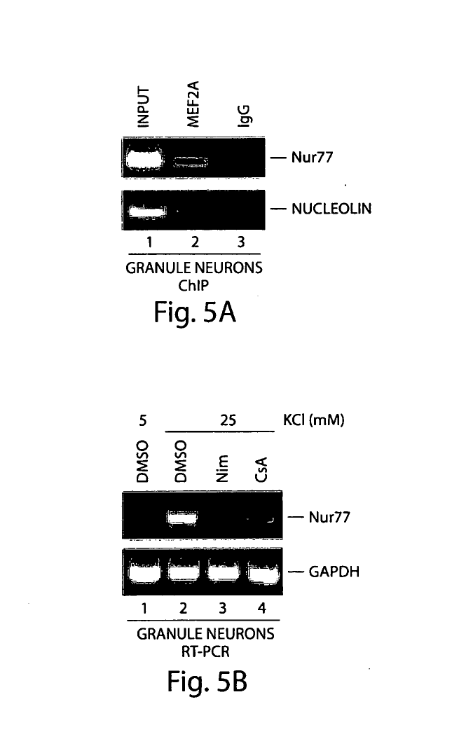

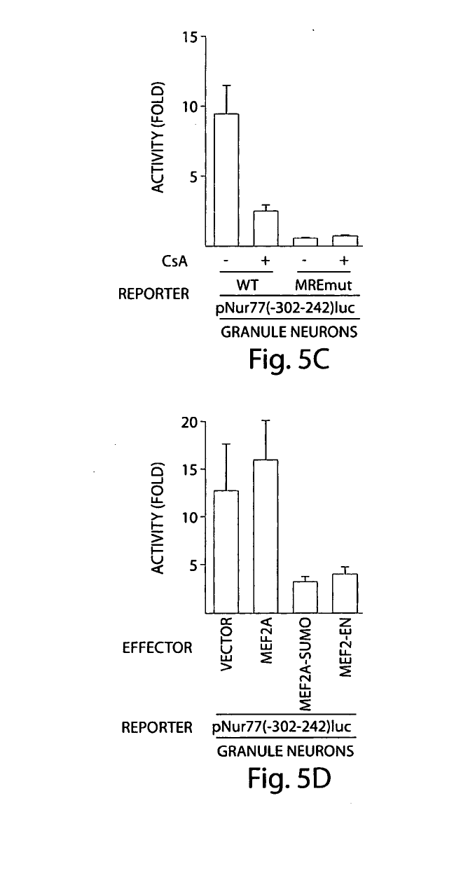

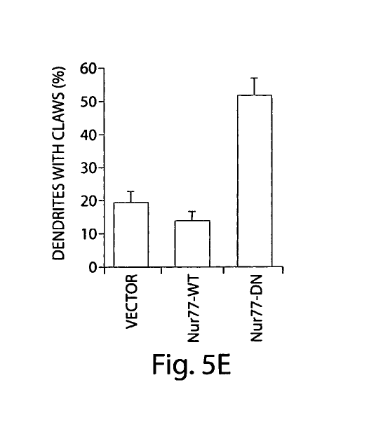

The mechanism by which sumoylated MEF2A promotes dendritic claw differentiation was determined. Transcription of the gene encoding the transcription factor Nur77 is induced by a calcineurin-MEF2 signaling pathway in immune cells. Endogenous MEF2A was found to occupy the endogenous Nur77 promoter in granule neurons (FIG. 5A). Nur77 mRNA abundance and Nur77 promoter-mediated transcription increased in depolarized neurons in a VSSC- and calcineurin-dependent manner (FIGS. 5B and 5C). Both MEF2A-SUMO and MEF2-EN inhibited depolarization-induced Nur77 transcription (FIG. 5D). In cerebellar slices, expression of a dominant interfering form of Nur77 increased the number of dendritic claws (FIG. 5E). Thus, Nur77 represents a MEF2A target gene whose repression by sumoylated MEF2A contributes to dendritic claw differentiation.

These findings indicate that the transcription factor MEF2A plays a key role in the morphogenesis of granule neuron dendritic claws in the cerebellar cortex. The modifications of MEF2A required for postsynaptic differentiation occur within a phosphorylation-regulated sumoylation-acetylation switch (SAS) peptide motif that is conserved in all major MEF2 isoforms, except MEF2B, as well as several other transcription factor families. Thus, a phosphorylation-dependent switch between sumoylation and acetylation in transcription factors play a role in signal-regulated transcription and regulate diverse biological processes, including synapse development and plasticity.

### Example 6

### PIASx is a MEF2 SUMO E3 Ligase that Promotes Postsynaptic Dendritic Morphogenesis

Postsynaptic morphogenesis of dendrites is essential for the establishment of neural connectivity in the brain, but the mechanisms that govern postsynaptic dendritic differentiation remain poorly understood. Sumoylation of the transcription factor MEF2A promotes the differentiation of postsynaptic granule neuron dendritic claws in the cerebellar cortex. The protein PIASx was identified as a MEF2A SUMO E3 ligase that represses MEF2-dependent transcription in neurons. Gain-of-function and genetic knockdown experiments in rat cerebellar slices and in the postnatal cerebellum in vivo revealed that PIASx drives the differentiation of granule neuron dendritic claws in the cerebellar cortex. MEF2A knockdown suppresses PIASxinduced dendritic claw differentiation, and expression of sumoylated MEF2A reverses PIASx knockdown-induced loss of dendritic claws. These findings define the PIASx-MEF2 sumoylation signaling link as a key mechanism that orchestrates postsynaptic dendritic morphogenesis, and identifies novel functions for SUMO E3 ligases in brain development and plasticity.

The transcription factor myocyte enhancer factor 2A (MEF2A) plays a critical role in postsynaptic dendritic morphogenesis in the brain. Genetic knockdown of MEF2A by RNA interference (RNAi) in rat cerebellar slices and in the developing postnatal rat cerebellum in vivo revealed an essential function for MEF2A in granule neuron dendritic claw differentiation. A SUMO-modified form of MEF2A that acts as a transcriptional repressor induces postsynaptic dendritic differentiation. Experiments were carried out to elucidate the identity of the enzyme that stimulates MEF2A sumoylation and thereby drives postsynaptic dendritic morphogenesis.

Sumoylation, the covalent linkage of a small ubiquitin-related modifier (SUMO) to the camine of lysine residues in target proteins, requires the activities of three sets of enzymes. SUMO is first attached to the bipartite SUMO-activating enzyme Aos1/Uba2 (E1) in an ATP-dependent manner, followed by transfer of SUMO to the SUMO-conjugating enzyme Ubc9 (E2). In turn, Ubc9 catalyzes the transfer of SUMO to a substrate protein, a reaction that is facilitated by a SUMO E3 ligase. The PIAS proteins form the largest family of SUMO E3 ligases. These proteins were originally isolated based on their ability to inhibit STAT proteins, hence the name protein inhibitors of activated STAT (PIAS). The PIAS proteins were found to encode SUMO E3 ligase activity. Prior to the data described herein, the biological functions of the PIAS SUMO ligases in the nervous system were unknown.

PIASx, a MEF2A SUMO E3 ligase, promotes dendritic claw differentiation in the cerebellar cortex. By controlling MEF2A sumoylation and consequent postsynaptic dendritic differentiation, PIASx plays a pivotal role in the establishment of neuronal connectivity in the mammalian brain.

The following reagents and methods were used to generate the data described below.

### Plasmids and Antibodies

The pBJ5-FLAG-HDAC4 expression plasmid was a gift of Dr. Stuart Schreiber. The pCDNA3-HA-Calcineurin A* was generated by cloning the cDNA encoding constitutively active calcineurin A into pCDNA3. The MEF2A and GAL4-MEF2A expression plasmids including MEF2A and GAL4-MEF2A sumoylation mutants, pCDNA3-HA-SUMO1, and luciferase and renilla reporter constructs are described (Shalizi et al., 2006, Science 311:1012-1017).

The PIASx RNAi plasmids were generated by cloning the following oligonucleotides into pBS/U6 or pBS/U6-cmv-GFP, where the underlined text indicates the targeted sequence of PIASx: piasx1 5′-AACAGAAGCGCCCTGGACGCTTCAAGCTTGCGTCCAGGGCGCTTCTGTTCTTTTT G3′ (SEQ ID NO: 1); piasx2 5′-GGGTTCTCATGTATCAGCCATACAAGCTTTGGCTGATACATGAGAACCCCTTTTT G3′ (SEQ ID NO: 2). The RNAi-resistant PIASx-Res construct was generated by QuikChange site directed mutagenesis (Stratagene) according to the manufacturer's protocol, and incorporated the following silent mutations indicated by lower case letters: 5′-GTg CTa ATG TAc CAa-3′ (SEQ ID NO: 3).

The PIASx antibodies were used to characterize the enzyme. The FLAG monoclonal antibody was purchased from Sigma. The HA polyclonal, MEF2 polyclonal, and GAL4 monoclonal antibodies were purchased from Santa Cruz. The GFP polyclonal antibody was purchased from Molecular Probes. The HA monoclonal antibody was purchased from Covance. The ERK1/2 antibody was purchased from Promega. The MEF2A-pS408 polyclonal antibody used to characterize the factor.

### Cell Culture and Transfections

Cultures of primary granule neurons were isolated from P6 Long-Evans rats using known methods. Granule neurons were maintained in full medium (BME+10% calf serum (Hyclone), 1 mM each penicillin, streptomycin and L-glutamine and 25 mM KCl). Granule neurons were transfected in DMEM by DNA-calcium phosphate precipitation using standard methods.

293T cells were maintained in DMEM supplemented with 10% calf serum, and 1 mM each of penicillin, streptomycin and L-glutamine. 293T cells were transfected by DNA-calcium phosphate precipitation using known methods. Medium was replaced 24 hours after transfection, and cells were harvested 48 hours after transfection for in vivo sumoylation assays, co-immunoprecipitation studies or luciferase-reporter assays, and 72-96 hours after transfection for RNAi studies.

### Protein Immunoprecipitation and Sumoylation Assays

In vivo sumoylation assays were performed as described previously. Briefly, HEK293T cells cotransfected with expression plasmids for full-length MEF2A or GAL4-MEF2A, HA-SUMO1 and other proteins as indicated, were lysed in RIPA buffer (150 mM NaCl, 10 mM Na2HPO4 pH 7.2, 2 mM EDTA, 50 mM NaF, 1 mM NaVO4, 1% NP-40, 0.1% SDS, 0.75% sodium deoxycholate, 1 mM PMSF, 10 mM N-ethylmaleimide, 10 μg/ml aprotinin) and pre-cleared with protein A-sepharose beads. Five percent of this starting material was retained for detection of input proteins and the remainder was subjected to immunoprecipitation overnight at 4° C. For experiments using full length MEF2A, a MEF2 polyclonal antibody (Santa Cruz Biotechnology) was used together with protein A-sepharose beads, and for experiments using GAL4-MEF2A, an agarose-conjugated GAL4 monoclonal antibody (Santa Cruz Biotechnology) was used. Immune complexes were washed 5 times with RIPA buffer at 4° C. and resuspended in Laemmli buffer. Immune complexes and input samples were subjected to SDSPAGE, transferred to nitrocellulose membranes and probed with the indicated antibodies.

Coimmunoprecipitation experiments were performed as follows. Briefly, 293T cells cotransfected with expression plasmids for FLAG-PIASx and MEF2A-WT or MEF2A-S408A were lysed in co-IP buffer (150 mM NaCl, 50 mM TrisHCl pH 7.5, 1 mM EDTA, 50 mM NaF, 1 mM NaVO4, 1% NP-40, 1 mM PMSF, 10 mM N-ethylmaleimide, 10 μg/ml aprotinin) and pre-cleared with protein G-sepharose beads. Five percent of this starting material was retained for detection of input proteins and the remainder was subjected to immunoprecipitation with anti-FLAG antibodies for 4 hours at 4° C. Immune complexes were bound to protein G-sepharose beads for 1 hour at 4° C., washed twice with co-IP buffer, once with PBS (pH 7.4), and resuspended in Laemmli buffer. Immune complexes and input samples were subjected to SDS-PAGE, transferred to nitrocellulose membranes and probed with the indicated antibodies.

### Luciferase Assays

Luciferase assays were performed as described (Shalizi et al., 2006, Science 311: 1012-1017) with minor modifications. Granule neurons were transfected with the reporter constructs pNur77-luc or pNur77mut-luc and pRL-TK and the indicated hpRNA expression plasmids, and an expression construct for Bcl-XL. Granule neurons were switched from full medium to fresh BME supplemented with 5% calf serum (Hyclone) 72 hours after transfection and incubated overnight. 293T cells maintained as described were cotransfected with p5G4luc or pMEF2×3luc and pRL-TK reporter constructs and the indicated expression plasmids by DNA-calcium phosphate precipitation. Fresh growth media was added within 24 hours of transfection. Cells were lysed 48 hours after transfection. In both neurons and 293T cells, firefly-and renilla-luciferase activities were determined using a dual-luciferase assay kit (Promega) according to the manufacturers instructions.

### RT-PCR

RNA was prepared from 293T cells or granule neurons using TRIzol (Invitrogen) according to the manufacturer's instructions. Purified RNA was subjected to RT-PCR using the SuperScript II one-step RT-PCR system (Invitrogen) according to the manufacturer's protocol. Amplification conditions were as follows: cDNA synthesis for 30 minutes at 55° C. followed by 1 minute at 95° C. and 25 (GAPDH) or 30 (PIASx) cycles of amplification at 95° C. for 30 seconds, 55° C. for 30 seconds and 72° C. for 1 minute, with a final extension at 72° C. for 5 minutes. PCR products were separated by agarose gel eletrophoresis in 1× TAE. Primers for GAPDH have been described previously. Primers for PIASx were sense 5′-CCTTTGCCTGGCTATGCACC-3′ (SEQ ID NO: 4) and antisense 5′-CAGGACAAATCCAGGTGGGC-3′ (SEQ ID NO: 5).

### Cerebellar Slices

Slices were prepared and processed using known methods. Briefly, cerebella from postnatal day 9 or 10 rats were dissected in HHGN (2.5 mM HEPES, 35 mM glucose, 4 mM NaHCO3 diluted in Cellgro HBSS) and cut into 400 μm sagittal slices using a tissue chopper (McIllwain), and transferred to a porous membrane (Millicell-CM Low Height Culture Plate Insert) that allows for an air-media interface, and maintained in MEM supplemented with 25% horse serum, 2.5% 10× Cellgro HBSS, 1% Gibco Penicillin-Streptomycin-Glutamine, 12.5 mM HEPES, and 22 mM glucose. At DIV4, slices were transfected using biolistics (Helios Gene Gun, BioRad). At DIV8, slices were fixed in 4% paraformaldehyde and permeabilized in 0.4% Triton X-100 in PBS. Slices were incubated with a rabbit GFP antibody (Molecular Probes A6455) at 1:500 in 1% goat serum, 0.05% BSA, 0.025% sodium azide, 0.4% Triton X-100 in PBS overnight at 4° C., and then with goat anti-rabbit secondary antibody conjugated with Cy2 or Cy3 (Amersham) at 1:500 for 2 hours at RT. Nuclei were stained with the DNA dye bisbenzimide (Hoechst 33258).

Microscopy was carried out using standard methods. For confocal imaging of cerebellar slices, Z series (0.5 μm) of images of transfected granule neurons were obtained at 60× magnification on a Nikon TE2000-U spinning disc confocal microscope. Two-dimensional reconstruction of Z series images was then performed using a maximum brightness projection algorithm (Volocity imaging software). Images of transfected granule neurons were analyzed using SPOT software for dendritic length, number of primary dendrites, and number of branches per primary dendrite as described.

### In Vivo Electroporation in the Postnatal Cerebellum

Rat pups (P3) were subjected to in vivo electroporation and analyzed by immunohistochemical analysis at P12 as described (Shalizi et al., 2006, Science 311:1012-1017).

### Cerebellar and Cortical Lysates

The cerebellum and cerebral cortex were dissected from P6, P10, and P14 rat pups in HHGN. Following isolation, these structures were transferred to lysis buffer (50 mM Tris pH 7.5, 150 mM NaCl, 2 mM EDTA, 1% Triton X-100, 1 mM DTT, 50 mM NaF, 1 mM sodium orthovanadate, 3 μg/ml aprotinin, 1 μg/ml leupeptin, 2 μg/ml pepstatin) and homogenized using a Brinkmann Polytron homogenizer (Kinematica). After ten minutes, the homogenates were spun down at 14,000 rpm and supernatant was collected.

### Results

### PIASx is a MEF2A SUMO E3 Ligase

The finding that sumoylation of MEF2A plays a key role in postsynaptic dendritic morphogenesis raised the fundamental issue of identifying the SUMO E3 ligase that stimulates MEF2A sumoylation and thereby promotes postsynaptic dendritic differentiation. The PIAS proteins comprise the largest family of SUMO E3 ligases. Although the PIAS proteins are expressed in the nervous system, prior to the data described herein, their functions in the nervous system were largely unknown.

Using a candidate approach, it was determined if a PIAS protein might promote SUMO-modification of MEF2A in a sumoylation assay in cells. MEF2A and HA-tagged SUMO were coexpressed alone or together with members of the PIAS family of proteins (PIAS1, PIAS3, PIASxα, PIASxβ, PIASy) in 293T cells. Since class IIa histone deacetylases (HDACs) interact with MEF2 proteins and are associated with SUMO E3 ligase activity, the ability of HDAC4 to stimulate MEF2A sumoylation was examined. Lysates of transfected cells were subjected to immunoprecipitation of MEF2A followed by immunoblotting with an antibody to HA to detect SUMO-modified MEF2A. Among the different SUMO E3 ligases, only PIASxα and PIASxβ efficiently increased the level of SUMO-modified MEF2A. PIASxα and PIASxβ, which represent the products of spliced mRNAs encoded by the same gene, promoted MEF2A sumoylation to a similar extent. For the sake of clarity in the remainder of the study, the two isoforms collectively will be referred to as PIASx.

The potency of the different PIAS proteins to promote the sumoylation of MEF2A was examined by transfecting increasing amounts of PIAS1, PIAS3, PIASx, and PIASy in cells. In these experiments, PIASx most robustly induced MEF2A sumoylation. By contrast, expression of PIAS proteins other than PIASx often reduced the amount of SUMO-modified MEF2A. In coimmunoprecipitation experiments, PIASx, PIAS1, PIAS3, and PIASy interacted with MEF2A. These results are consistent with the possibility that PIAS proteins other than PIASx may have titrated critical co-factors of the machinery necessary for MEF2A sumoylation.

Sumoylation of MEF2A represses MEF2-dependent transcription. The effect of PIASx expression on MEF2-dependent transcription in cells was examined. 293T cells were transfected with a luciferase reporter gene controlled by MEF2-response elements (MRE-luciferase) together with a PIASx expression plasmid or its control vector. In these experiments, PIASx potently inhibited MRE-luciferase reporter gene expression. Thus, consistent with its ability to stimulate MEF2A sumoylation, PIASx represses MEF2-dependent transcription.

The sumoylation of MEF2A occurs on Lysine 403, which is part of a conserved peptide motif within the MEF2 repressor domain. Importantly, efficient sumoylation of MEF2A is dependent on the phosphorylation of MEF2A at the nearby site of Serine 408. It was determined if PIASx induces sumoylation of MEF2A at Lysine 403 and whether the PIASx-induced MEF2A sumoylation is controlled by Serine 408 phosphorylation. In assays of sumoylation, while PIASx stimulated the sumoylation of wild type MEF2A, PIASx failed to trigger the sumoylation of a MEF2A mutant in which Lysine 403 was replaced with arginine (MEF2AK403R). In other experiments, although PIASx interacted with a MEF2A mutant in which Serine 408 was replaced with alanine (MEF2AS408A) as efficiently as with wild type MEF2A, PIASx failed to induce the robust sumoylation of MEF2AS408A. The ability of PIASx to induce the sumoylation of wild type MEF2A was also significantly reduced upon coexpression of the activated form of the phosphatase calcineurin, which induces the dephosphorylation of MEF2A at Serine 408. These results suggest that PIASx induces the sumoylation of MEF2A at Lysine 403 in a Serine 408 phosphorylation-dependent manner. Collectively, evidence suggests that PIASx represents a bona-fide MEF2A SUMO E3 ligase.

### PIASx Promotes Dendritic Claw Differentiation in the Cerebellar Cortex

The function of PIASx in neurons was examined beginning with characterizing the expression of PIASx in granule neurons in the cerebellum. PIASx mRNA and protein were detected in primary cerebellar granule neurons by RT-PCR and immunoblotting analyses. Immunohistochemical analysis of the developing rat cerebellar cortex revealed expression of PIASx in both Purkinje and granule neurons within the internal granule layer (IGL). PIASx expression was present in the cerebellar cortex in rat pups during the first and second week postnatally. This pattern of expression overlaps with that of MEF2A. Since PIASx acts as a MEF2A SUMO E3 ligase, the overlapping pattern of PIASx and MEF2A expression in the cerebellar cortex suggested that PIASx might regulate MEF2A function in neurons.

To determine the role of endogenous PIASx in the control of MEF2-regulated transcription in neurons, a plasmid-based method of RNAi interference (RNAi) was used to acutely knockdown PIASx. Plasmids that encode hairpin RNAs (hpRNAs) targeting two distinct regions of PIASx (U6/piasx I and U6/piasx2) were constructed. Expression of each piasx hairpin RNA but not a control-scrambled hairpin RNA induced the efficient knockdown of PIASx protein in cells. PIASx RNAi induced the specific knockdown of PIASx but not the related protein PIAS1. In addition, PIASx RNAi reduced endogenous PIASx immunoreactivity in primary granule neurons obtained with the antibody used in the methods described above. Finally, PIASx RNAi reduced efficiently the expression of both PIASxαand PIASxβin cells. Collectively, these experiments indicate that PIASx RNAi reduces the expression of PIASx in cells and primary neurons.

Primary rat cerebellar granule neurons were transfected with the U6/piasx1, U6/piasx2, or control U6 RNAi plasmid together with a luciferase reporter gene controlled by the Nur77 promoter containing two MEF2 response elements (MREs). Nur77 is a direct repressed target gene of sumoylated MEF2A in granule neurons, whose repression promotes postsynaptic dendritic differentiation in the cerebellar cortex. Knockdown of PIASx, using either the U6/piasx1 or U6/piasx2 RNAi plasmid, significantly increased the level of Nur77 promoter-mediated transcription in granule neurons. Expression of the control-scrambled hairpin RNA had no effect on the level of the Nur77 promoter. In other experiments, we found that PIASx RNAi failed to induce the expression of a luciferase reporter gene driven by a Nur77 promoter-containing mutant MREs. These results demonstrate that PIASx knockdown derepresses Nur77 promoter-mediated transcription in an MRE-dependent manner. Together, these experiments support the conclusion that PIASx represses MEF2-dependent transcription in primary neurons.

Next, the role of PIASx in neuronal morphogenesis was examined. It has recently been shown that sumoylation of MEF2A promotes the differentiation of dendritic claws in granule neurons of the developing cerebellum. To determine PIASx function in dendritic morphogenesis in the cerebellar cortex, PIASx knockdown was induced in rat cerebellar slices. Using a biolistics approach, cerebellar slices prepared from postnatal day 10 (P10) rat pups were transfected with the U6/piasx1, U6/piasx2, or control U6 plasmid that also encoded green fluorescent protein (GFP) bicistronically. The U6/piasx-cmvGFP RNAi constructs were confirmed to induce the knockdown of PIASx. Four days later, cerebellar slices were subjected to immunohistochemistry with an antibody to GFP to visualize transfected neurons within the cerebellar cortex. Granule neurons in the IGL were found with their typical small cell body and associated parallel fiber axons. The dendrites of control U6-transfected neurons often harbored dendritic claws. Dendritic claws were identified on the basis of classic descriptions as dendritic structures that are present at the end of dendrites, having cuplike or sicklelike appearance with inner serrated or undulating surfaces. Dendritic claws visualized in cerebellar slices are enriched with postsynaptic protein PSD95 puncta, indicating that dendritic claws represent sites of postsynaptic differentiation.

PIASx knockdown neurons had significantly fewer dendritic claws than control U6-transfected neurons, and dendrites of PIASx knockdown neurons typically displayed tapered ends. There was a 50 and 70 percent reduction in the number of dendritic claws in cerebellar slices transfected with the piasx1 and piasx2 RNAi plasmids respectively. The PIASx knockdown-induced dendritic claw phenotype was not due to impaired dendritic growth or branching, as PIASx RNAi did not lead to a reduction in dendritic length or the number of branches in cerebellar slices. Taken together, these results suggest that PIASx plays a critical role in the differentiation of granule neuron dendritic claws.

To rule out the possibility that the PIASx knockdown-induced dendritic claw phenotype was the result of activation of the RNAi machinery per se, rescue experiments were performed. An expression plasmid encoding wild type PIASx protein was constructed using cDNA designed to be resistant to piasx2 hpRNAs (PIASx-Res). While expression of piasx1 hpRNAs robustly induced knockdown of PIASx-Res, piasx2 hpRNAs failed to effectively trigger knockdown of PIASx-Res. In cerebellar slices, while PIASx RNAi led to a significant reduction of dendritic claw number in granule neurons, expression of PIASx-Res reversed the piasx2 RNAi induced dendritic phenotype, restoring the number of claws to nearly 80 percent of control U6 transfected cerebellar slices. These results indicate that the PIASx RNAi-induced dendritic claw phenotype is the result of specific knockdown of PIASx rather than off-target effects of RNAi.

Having identified a requirement for endogenous PIASx in the differentiation of the postsynaptic dendritic claws in granule neurons, the effect of increasing the levels of PIASx above its endogenous levels on dendritic claw differentiation was examined. Cerebellar slices were transfected with the PIASx expression plasmid or its control vector together with a GFP expression plasmid. In these experiments, slices prepared from P9 rat pups were used instead of P10 in order to have a lower baseline of dendritic claw number in control-transfected slices. Expression of PIASx robustly increased the number of dendritic claws in IGL granule neurons, suggesting that PIASx stimulates dendritic claw differentiation. Thus, on the basis of both loss-of-function knockdown experiments and gain-of-function experiments, PIASx plays a key role in dendritic claw morphogenesis in the cerebellar cortex.

To establish the importance of PIASx in granule neuron dendritic development in vivo, the knockdown of PIASx was induced in the postnatal rat cerebellum using electroporationmediated gene transfer. The U6/piasx1-cmvGFP or control U6-cmvGFP RNAi plasmid was injected into the cerebellar cortex of P3 rat pups and examined the morphology of IGL granule neurons 9 days later in these animals at P12. In control-transfected cerebella, granule neurons had well-defined dendritic claws at the ends of dendrites. In contrast, in animals transfected with the PIASx RNAi plasmid granule neurons displayed tapered or bulbous dendritic ends. Quantitative analyses revealed that PIASx knockdown led to a significant reduction in the number of dendritic claws. These results show that endogenous PIASx plays a critical role in the differentiation of dendritic claws in vivo in the postnatal rat cerebellar cortex.

### PIASx Drives Dendritic Claw Differentiation via MEF2 Sumoylation

It was hypothesized that the novel function of PIASx in dendritic claw differentiation is mediated via sumoylation of MEF2A. It was determined if the gain-of-function effect of PIASxinduced dendritic claw differentiation requires the presence of MEF2A protein. Knockdown of MEF2A on its own as expected dramatically reduced the number of dendritic claws in rat cerebellar slices, an effect that has been demonstrated to be secondary to loss of the sumoylated transcriptional repressor form of MEF2A. Knockdown of MEF2A completely suppressed the ability of PIASx overexpression to increase the number of dendritic claws in cerebellar slices. These results are consistent with the conclusion that MEF2A acts downstream of PIASx in dendritic claw differentiation.

The effect of expression a MEF2A-SUMO fusion protein on the dendritic claw phenotype induced by PIASx knockdown was examined. The MEF2A-SUMO fusion protein mimics the effect of SUMO that is covalently linked to MEF2A on the native lysine and thus acts as a transcriptional repressor that promotes postsynaptic dendritic claw differentiation. Expression of MEF2A-SUMO, but not MEF2A, robustly increased the number of dendritic claws in the background of PIASx RNAi. Thus, sumoylated MEF2A suppresses the PIASx knockdown-induced dendritic claw phenotype.

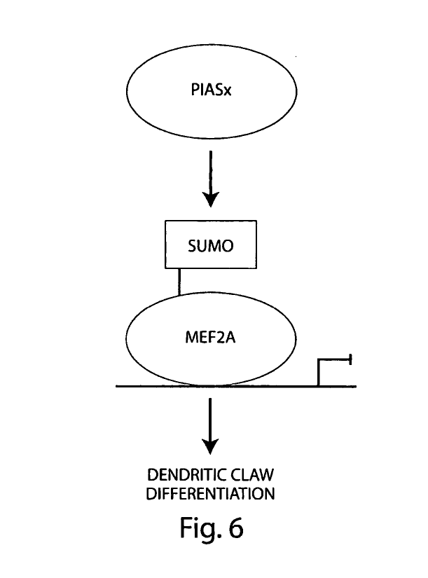

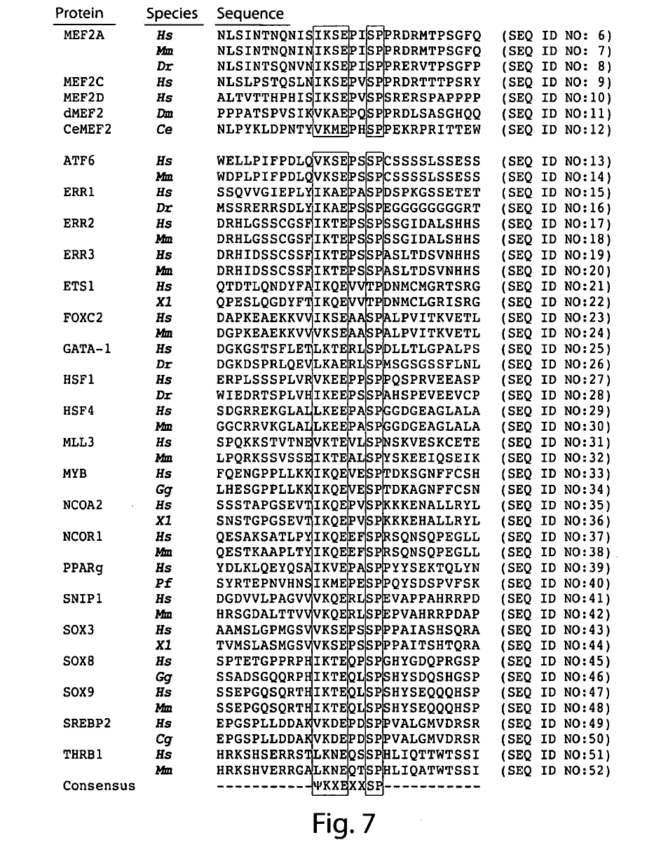

Sumoylated MEF2A drives dendritic claw differentiation via repression of the orphan nuclear receptor Nur77. The effect of expression of a dominant interfering form of Nur77 (DN Nur77) on dendritic claw differentiation in the background of PIASx RNAi in rat cerebellar slices was examined. Expression of DN Nur77, but not the wild type Nur77 (WT Nur77), significantly increased the number of dendritic claws in the background of PIASx RNAi. Thus, Nur77 inhibition mimicked the ability of sumoylated MEF2A to suppress the PIASx knockdown-induced dendritic claw phenotype. Collectively, these results support the conclusion that by acting as a MEF2A SUMO E3 ligase, PIASx promotes the morphogenesis of granule neuron dendritic claws in the cerebellar cortex (FIG. 6).

PIASx was identified as a MEF2 SUMO E3 ligase that promotes dendritic claw differentiation in the cerebellar cortex. Among the PIAS family of proteins, only PIASx stimulates the robust sumoylation of MEF2A and thereby represses MEF2-dependent transcription. PIASx induces MEF2A sumoylation at the key regulatory site of Lysine 403 in a Serine 408 phosphorylation-dependent manner. PIASx overexpression and inhibition studies in rat cerebellar slices and in vivo in the postnatal cerebellum demonstrate a function for PIASx in the differentiation of granule neuron dendritic claws in the cerebellar cortex, and expression of sumoylated MEF2A or inhibition of the sumoylated-MEF2A-repressed target gene Nur77 restores the appearance of dendritic claws in the background of PIASx knockdown. These data indicate that PIASx increases dendritic claw number via MEF2 sumoylation. Identification of PIASx as a major SUMO E3 ligase for the transcription factor MEF2 indicates that PIASx regulates the establishment and refinement of neural connectivity in the brain.

SUMO proteases inhibit the sumoylation of MEF2A at Lysine 403 and thereby regulate dendritic claw differentiation. MEF2 proteins including MEF2A, MEF2C, and MEF2D are widely expressed in the developing brain, and MEF2A and MEF2D are involved in the control of synapse number in hippocampal neurons. All MEF2 proteins except MEF2B are covalently conjugated with SUMO at a key regulatory site corresponding to Lysine 403 in MEF2A. PIASx can also function as a SUMO E3 ligase in the sumoylation of other MEF2 proteins. By sumoylating MEF2A or other MEF2 proteins, PIASx plays a role in postsynaptic dendritic development in diverse regions of the brain. In addition to its expression in the developing cerebellar cortex, PIASx is expressed elsewhere in the brain including the cerebral cortex and hippocampus. The PIASxMEF2 signaling link therefore plays a role in the refinement of postsynaptic dendritic morphology and synaptic plasticity.

PIASx-induced sumoylation of MEF2A at Lysine 403 is dependent on the phosphorylation of MEF2A at the nearby site of Serine 408. The Serine 408 phosphorylation does not appear to recruit PIASx, as MEF2A interacts with PIASx regardless of the Serine 408 phosphorylation status. Thus, phosphorylation may render the Lysine 403 peptide a better substrate for the PIASx-induced sumoylation.

The PIASxMEF2 signaling connection described herein indicates that PIASx controls dendritic morphogenesis, and MEF2 sumoylation as it relates to MEF2's function in neuronal survival. Outside the brain, PIASx regulates the functions of MEF2 in muscle differentiation and muscle fiber type switching.

What is claimed is:

1. A method of identifying a candidate compound for modulating synapse formation, said method comprising: (a) contacting a cell expressing a sumoylation-acetylation switch (SAS) peptide motif gene with a candidate compound and (b) measuring the level of Serine408 (Ser408) phosphorylation of the SAS peptide motif in said cell, wherein a modulation in said phosphorylation level in the presence of said compound compared to that in the absence of said compound indicates that said compound modulates synapse number or differentiation.

2. The method of claim 1, wherein said synapse differentiation is dendritic claw differentiation.

3. The method of claim 1, wherein said candidate compound increases the level of Ser408 phosphorylation thereby increasing synapse number or differentiation.

4. The method of claim 1, wherein said candidate compound reduces the level of Ser408 phosphorylation thereby reducing synapse number or differentiation.

5. The method of claim 1, wherein said SAS peptide motif gene is a myocyte enhancer factor 2A (MEF2A) gene.

6. The method of claim 1, wherein said SAS peptide motif gene construct encodes a chimeric polypeptide, said polypeptide comprising a SAS peptide and a heterologous peptide.

7. The method of claim 1, wherein step (b) further comprises measuring the level of sumoylation, acetylation, or both at the Lys403 residue in said SAS motif peptide.

8. The method of claim 1, wherein step (b) further comprises measuring the expression or activity level of Nur77.

9. The method of claim 8, wherein said measuring comprises measuring the level of Nur77mRNA.

10. A method for identifying a candidate compound for modulating synapse formation, said method comprising: (a) contacting a cell expressing a SAS peptide motif gene with a candidate compound and (b) measuring the level of sumoylation at the Lys403 residue in the SAS peptide motif in said cell, wherein a modulation in said sumoylation levels in the presence of said compound compared to that in the absence of said compound indicates that said compound modulates synapse number or differentiation.

11. The method of claim 10, wherein said candidate compound increases the level of sumoylation at the Lys403 residue and is identified as a compound that increases synapse number, formation, or maturation.

12. The method of claim 10, wherein said candidate compound reduces the level of sumoylation at the Lys403 residue thereby reducing synapse number or formation.

13. The method of claim 10, wherein said SAS peptide motif gene is a MEF2A gene.

14. The method of claim 10, wherein said SAS peptide motif gene is a construct that encodes a polypeptide, said polypeptide comprising a SAS peptide motif and a heterologous gene.

15. The method of claim 10, wherein step (b) further comprises measuring the level of Ser408 phosphorylation, acetylation at the Lys403 residue, or both in said SAS motif peptide.

16. The method of claim 10, wherein step (b) further comprises measuring the expression or activity level of Nur77.

17. The method of claim 16, wherein said measuring comprises measuring the level of Nur77mRNA.

18. A method for identifying a candidate compound for modulating synapse formation, said method comprising: (a) contacting a cell expressing a SAS peptide motif gene with a candidate compound and (b) measuring the level of acetylation at the Lys403 residue in the SAS peptide motif in said cell, wherein a modulation in acetylation levels in the presence of said compound compared to that in the absence of said compound indicates that said compound modulates synapse number or formation.

19. The method of claim 18, wherein said candidate compound increases the levels of acetylation at the Lys403 residue thereby reducing synapse number or formation.

20. The method of claim 18, wherein said candidate compound reduces the levels of acetylation at the Lys403 residue thereby increasing synapse number or formation.

21. The method of claim 18, wherein said SAS peptide motif gene is a MEF2A gene.

22. The method of claim 18, wherein said SAS peptide motif gene is a construct encoding a polypeptide, said polypeptide encoding a SAS peptide motif and a heterologous gene.

23. The method of claim 18, wherein step (b) further comprises measuring the level of Ser408 phosphorylation, sumoylation at the Lys403 residue, or both in said SAS motif peptide.

24. The method of claim 18, wherein step (b) further comprises measuring the expression or activity level of Nur77.

25. The method of claim 24, wherein said measuring comprises measuring the level of Nur77mRNA.

26. The method of claim 1, 10, or 18, wherein said cell is a mammalian cell.

27. The method of claim 26, wherein said cell is a rodent or human cell.

28. The method of claim 1, 10, or 18, wherein said cell is a neural cell.

29. The method of claim 28, wherein said neural cell is a cerebellar granule neuron cell.

30. A method of modulating synapse formation by contacting a neural cell with an agent that modulates the level of Ser408 phosphorylation in the SAS peptide motif of MEF2A in said cell.

31. The method of claim 30, wherein said neural cell is a cerebellar granule neuron cell.

32. The method of claim 30, wherein said agent a small molecule inhibitor or an RNA interfering molecule.

33. The method of claim 30, wherein said agent increases Ser408 phosphorylation in the SAS peptide motif of MEF2A in said cell, thereby increasing synapse number or formation.

34. The method of claim 33, wherein said agent is a phosphatase inhibitor.

35. The method of claim 34, wherein said phosphatase inhibitor is cyclosporin A or FK506.

36. The method of claim 30, wherein said agent reduces Ser408 phosphorylation in the SAS peptide motif of MEF2A in said cell, thereby reducing synapse number or formation.

37. The method of claim 36, wherein said agent is a kinase inhibitor or an agent that increases the activity of a phosphatase.

38. The method of claim 30, further comprising contacting said cell with an agent that modulates the levels of acetylation at the Lys403 residue in the SAS peptide motif of MEF2A in said cell.

39. The method of claim 30, further comprising contacting said cell with an agent that modulates the levels of sumoylation at the Lys403 residue in the SAS peptide motif of MEF2A in said cell.

40. A method of modulating synapse formation by contacting a neural cell with an agent that modulates the level of sumoylation at the Lys403 residue in the SAS peptide motif of MEF2A in said cell.

41. The method of claim 40, wherein said neural cell is a cerebellar granule neuron cell.

42. The method of claim 40, wherein said agent a small molecule inhibitor or an RNA interfering molecule.

43. The method of claim 40, wherein said agent reduces said level of sumoylation in said cell, thereby reducing synapse number or formation.

44. The method of claim 43, wherein said agent reduces the expression or activity level of a SUMO protease, the SUMO conjugating enzyme Ubc9, or a SUMO E3 ligase.

45. The method of claim 44, wherein said agent is N-ethylmaleimide.

46. The method of claim 44, wherein said agent is a SUMO-removing isopeptidase.

47. The method of claim 40, wherein said agent increases said level of sumoylation in said cell, thereby increasing synapse formation.

48. The method of claim 47, wherein said agent reduces the expression or activity level of a histone acetyl transferase enzyme.

49. The method of claim 48, wherein said agent is curcumin or a derivative thereof.

50. The method of claim 48, wherein said agent is a HAT inhibitor.

51. The method of claim 49 or 50, wherein said agent reduces the level of acetylation at said Lys403 residue.

52. The method of claim 47, wherein said agent is nimodipine.

53. The method of claim 47, wherein said agent is a voltage-sensistive calcium channel (VSCC) or calcineurin inhibitor.

54. The method of claim 53, wherein said calcineurin inhibitor is Cyclosporin A (CsA).

55. A method of modulating synapse formation by contacting a neural cell with an agent that modulates the level of acetylation at the Lys403 residue in the SAS peptide motif of MEF2A in said cell.

56. The method of claim 55, wherein said neural cell is a granule neuron.

57. The method of claim 55, wherein said agent a small molecule inhibitor or an RNA interfering molecule.

58. The method of claim 55, wherein said agent reduces said level of acetylation in said cell, thereby increasing synapse formation.

59. The method of claim 58, wherein said agent is curcumin or a derivative thereof.

60. The method of claim 58, wherein said agent is a HAT inhibitor.

61. The method of claim 58, wherein said agent is nimodipine.

62. The method of claim 58, wherein said agent is a VSCC or calcineurin inhibitor.

63. The method of claim 62, wherein said calcineurin inhibitor is CsA.

64. The method of claim 55, wherein said agent increases said level of acetylation in said cell, thereby reducing synapse number or formation.

65. The method of claim 64, wherein said agent reduces the expression or activity level of a histone deacetylase (HDAC).

66. The method of claim 65, wherein said HDAC is class I HDAC, class II HDAC, or class III HDAC.

67. The method of claim 64, wherein said agent is trichostatin A, suberoylanilide hydroxamic acid (SAHA), pyroxamide, apicidin, depudecin, depsipeptide, oxamflatin, CI-994 (N-acetyl dinaline), m-Carboxy cinnamic acid bishydroxamic acid (CBHA), scriptaid, trapoxin, TPX-HA analogue (CHAP), or sirtinol.

68. The method of claim 64, wherein said agent increases the expression or activity level of a histone acetyltransferase.

69. A method of modulating synapse formation by contacting a neural cell with an agent that modulates the activity of a SUMO E3 ligase in said cell.

70. The method of claim 69, wherein said SUMO E3 ligase is PIASx.

71. A method of modulating synapse formation by contacting a neural cell with an agent that modulates the level of sumoylation at the Lys403 residue in the SAS peptide motif of MEF2A in said cell, wherein said agent is PIASx.

72. The method of claim 71, wherein said agent increases said level of sumoylation in said cell, thereby increasing synapse number or formation.

73. A method for identifying a candidate compound that modulates association of PIASx with MEF2A, said method comprising: (a) contacting a cell expressing a MEF2A gene with a candidate compound and (b) measuring the level of sumoylation at the Lys403 residue in the MEF2A gene in said cell, wherein a modulation in said sumoylation levels in the presence of said compound compared to that in the absence of said compound indicates that said compound modulates association of PIASx with MEF2A.

74. A method for identifying a candidate compound that modulates association of PIASx with MEF2A, said method comprising (a) contacting a cell expressing a MEF2A gene with a candidate compound and (b) measuring the association of PIASx with MEF2A in said cell, wherein a modulation in said association levels in the presence of said compound compared to that in the absence of said compound indicates that said compound modulates association of PIASx with MEF2A.

75. A method for identifying a candidate compound that modulates the enzymatic activity of PIASx, said method comprising (a) contacting a cell expressing PIASx with a candidate compound and (b) measuring the enzymatic activity of PIASx in said cell, wherein a modulation in said enzymatic activity levels in the presence of said compound compared to that in the absence of said compound indicates that said compound modulates enzymatic activity of PIASx.

76. The method of claim 30, 40, 55, 69, or 71, wherein said method reduces a symptom of a disorder selected from the group consisting of Alzheimer's disease, Parkinson's disease, stroke, multiple sclerosis, spinal cord injury, depression, schizophrenia, anxiety, Huntington's Disease, ALS, mental retardation (Down syndrome or Fragile X syndrome) and spinal muscular atrophy.

77. The use of an inhibitor of MEF2-dependent transcription in the manufacture of a medicament for increasing synapse number or differentiation.

78. The use of a PIASx activator in the manufacture of a medicament for increasing synapse number or differentiation.
